SEQUENCE LISTING (32 sequences)

SEQ ID NO: 1 - protein, 440 aa, Artificial Sequence
  Synthetic Peptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 214 aa, Artificial Sequence
  Synthetic Peptide
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 447 aa, Artificial Sequence
  Synthetic Peptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 218 aa, Artificial Sequence
  Synthetic Peptide
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 117 aa, Artificial Sequence
  Synthetic Peptide
EVQLVESGGGLVQPGGSLRLSCAASGFTFSDSWIHWVRQAPGKGLEWVAWISPYGGSTYYADSVKGRFTSADTSKNTAYLQMNSLRAEDTAVYYCARRHWPGGFDYWGQGTLVTVSA

SEQ ID NO: 6 - protein, 108 aa, Artificial Sequence
  Synthetic Peptide
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 7 - protein, 118 aa, Artificial Sequence [sequence omitted]

SEQ ID NO: 8 - protein, 122 aa, Artificial Sequence
  Synthetic Peptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTK

SEQ ID NO: 9 - protein, 108 aa, Artificial Sequence; identical to SEQ ID NO: 6
  Synthetic Peptide

SEQ ID NO: 10 - protein, 10 aa, Artificial Sequence
  Synthetic Peptide
  MOD_RES (6)..(6): Xaa is Asp or Gly
GFTFSXSWIH

SEQ ID NO: 11 - protein, 18 aa, Artificial Sequence
  Synthetic Peptide
  MOD_RES (4)..(4): Xaa is Ser or Leu
  MOD_RES (10)..(10): Xaa is Thr or Ser
AWIXPYGGSXYYADSVKG

SEQ ID NO: 12 - protein, 9 aa, Artificial Sequence
  Synthetic Peptide
RHWPGGFDY

SEQ ID NO: 13 - protein, 25 aa, Artificial Sequence [sequence omitted]

SEQ ID NO: 14 - protein, 13 aa, Artificial Sequence
  Synthetic Peptide
WVRQAPGKGLEWV

SEQ ID NO: 15 - protein, 32 aa, Artificial Sequence
  Synthetic Peptide
RFTISADTSKNTAYLQMNSLRAEDTAVYYCAR

SEQ ID NO: 16 - protein, 11 aa, Artificial Sequence
  Synthetic Peptide
WGQGTLVTVSA

SEQ ID NO: 17 - protein, 11 aa, Artificial Sequence
  Synthetic Peptide
  MOD_RES (5)..(5): Xaa is Asp or Val
  MOD_RES (6)..(6): Xaa is Val or Ile
  MOD_RES (7)..(7): Xaa is Ser or Asn
  MOD_RES (9)..(9): Xaa is Ala or Phe
  MOD_RES (10)..(10): Xaa is Val or Leu
RASQXXXTXXA

SEQ ID NO: 18 - protein, 7 aa, Artificial Sequence
  Synthetic Peptide
  MOD_RES (4)..(4): Xaa is Phe or Thr
  MOD_RES (6)..(6): Xaa is Tyr or Ala
SASXLXS

SEQ ID NO: 19 - protein, 9 aa, Artificial Sequence
  Synthetic Peptide
  MOD_RES (3)..(3): Xaa is Tyr, Gly, Phe, or Ser
  MOD_RES (4)..(4): Xaa is Leu, Tyr, Phe or Trp
  MOD_RES (5)..(5): Xaa is Tyr, Asn, Ala, Thr, Gly, Phe, or Ile
  MOD_RES (6)..(6): Xaa is His, Val, Pro, Thr, or Ile
  MOD_RES (8)..(8): Xaa is Ala, Trp, Arg, Pro, or Thr
QQXXXXPXT

SEQ ID NO: 20 - protein, 23 aa, Artificial Sequence
  Synthetic Peptide
DIQMTQSPSSLSASVGDRVTITC

SEQ ID NO: 21 - protein, 15 aa, Artificial Sequence
  Synthetic Peptide
WYQQKPGKAPKLLIY

SEQ ID NO: 22 - protein, 32 aa, Artificial Sequence
  Synthetic Peptide
GVPSRFSGSGSGTDFTLTISSLQPEDFATYYC

SEQ ID NO: 23 - protein, 11 aa, Artificial Sequence
  Synthetic Peptide
FGQGTKVEIKR

SEQ ID NO: 24 - protein, 10 aa, Artificial Sequence
  Synthetic Peptide
GFTFSDSWIH

SEQ ID NO: 25 - protein, 18 aa, Artificial Sequence
  Synthetic Peptide
AWISPYGGSTYYADSVKG

SEQ ID NO: 26 - protein, 11 aa, Artificial Sequence
  Synthetic Peptide
RASQDVSTAVA

SEQ ID NO: 27 - protein, 7 aa, Artificial Sequence
  Synthetic Peptide
SASFLYS

SEQ ID NO: 28 - protein, 9 aa, Artificial Sequence
  Synthetic Peptide
QQYLYHPAT

SEQ ID NO: 29 - protein, 118 aa, Artificial Sequence
  Synthetic Peptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 30 - protein, 11 aa, Artificial Sequence
  Synthetic Peptide
WGQGTLVTVSS

SEQ ID NO: 31 - protein, 447 aa, Artificial Sequence
  Synthetic Peptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 214 aa, Artificial Sequence
  Synthetic Peptide
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0239895 A1
(43) Pub. Date: Jul. 18, 2024

(54) COMBINATION THERAPY WITH A RAF INHIBITOR AND A PD-1 AXIS INHIBITOR

(71) Applicants: Genentech, Inc., South San Francisco, CA (US); Hanmi Pharm. Co., Ltd., Seoul (KR)

(72) Inventors: Darlene Dela Cruz, South San Francisco, CA (US); Shiva Malek, South San Francisco, CA (US); Ehud Segal, South San Francisco, CA (US); Ivana Yen Yen YEN, South San Francisco, CA (US); In Hwan BAE, Seoul (KR); Kwee Hyun SUH, Seoul (KR)

(21) Appl. No.: 18/554,236

(22) PCT Filed: Apr. 7, 2022
(86) PCT No.: PCT/US22/23775

§ 371 Date: Oct. 6, 2023

Related U.S. Application Data
(60) Provisional application No. 63/173,207, filed on Apr. 9, 2021.

(51) Int. Cl. C07K 16/28; A61K 31/519; A61K 39/00; A61P 35/00

(52) U.S. Cl. CPC C07K 16/2827; A61K 31/519; A61P 35/00; A61K 2039/505; A61K 2039/545

(57) ABSTRACT

A combination therapy comprising a RAF inhibitor and a PD-1 axis inhibitor is provided for the treatment of cancer characterized by a mutated MAPK signaling pathway.

### SEQUENCE LISTING

The present application includes a sequence listing in computer readable format submitted with this application through EFS-Web. Said sequence listing, created on Mar. 31, 2022, is 30,720 bytes in size. This sequence listing is incorporated by reference herein in its entirety.

### CROSS REFERENCE TO RELATED APPLICATION

This application claims priority to U.S. Provisional Application Ser. No. 63/173,207 filed on Apr. 9, 2021. The entire text of that provisional application is incorporated by reference into this application.

### FIELD OF THE DISCLOSURE

The field of the invention relates generally to cancer therapy with a combination of a RAF inhibitor and a PD-1 axis inhibitor.

### BACKGROUND

RAS genes are the most frequently mutated oncogenes in human cancer.

Among the RAS isoforms, KRAS is the most frequently mutated (86%), followed by NRAS (11%), which is predominantly mutated in cutaneous melanoma (28%). See: Cox A D, Fesik S W, Kimmelman A C, et al, “Drugging the undruggable RAS: Mission possible?”, Nat Rev Drug Discov 13:828-51, 2014; Hilmi Kodaz, Osman Kostek, Muhammet Bekir Hacioglu, et al., “Frequency of RAS Mutations (KRAS, NRAS, HRAS) in Human Solid Cancer”, EJMO 1:1-7, 2017; and Cancer Genome Atlas N, “Genomic Classification of Cutaneous Melanoma”, Cell 161:1681-96, 2015. Preclinical models of RAS-mutant driven cancers have demonstrated the role of KRAS and NRAS in tumor initiation and maintenance. To date, however, there has been limited clinical success in treating RAS-mutant tumors by targeting its downstream effector pathways, such as the inhibition of PI3K and MEK.

The RAF kinase family, which consist of three subtypes (A-RAF, B-RAF, C-RAF), is a key component of the MAPK signaling pathway downstream of RAS.

Mutations in RAF genes, particularly BRAF at codon V600, have been identified in various cancers, including malignant melanoma, colorectal, thyroid, and lung cancers. See Davies H, Bignell G R, Cox C, et al., “Mutations of the BRAF gene in human cancer”, Nature 417:949-54, 200. The BRAF V600 mutations enable BRAF to signal as a monomer, thereby constitutively activating the downstream MAPK signaling pathway.

The discovery of BRAF monomer inhibitors, such as, vemurafenib, dabrafenib, and encorafenib, has led to notable advances in the treatment of patients with BRAF^{V600}-mutant tumors; nevertheless, the durability of treatment response has been limited due to a variety of resistance mechanisms including BRAF amplification, BRAF splice variants and RAS mutations, that largely converge on BRAF dimerization and resistance to BRAF V600 monomer therapies. See Sullivan R J, Flaherty K T, “Resistance to BRAF-targeted therapy in melanoma” Eur J Cancer 49:1297-304, 2013. Furthermore, these BRAF^{V600} inhibitors have also been shown to paradoxically activate the MAPK signaling pathway in BRAF wild-type and KRAS-mutant cell lines, resulting in the dimerization of BRAF and CRAF, and activation of MEK and ERK signaling in a RAS-dependent manner. See: Heidorn S J, Milagre C, Whittaker S, et al., “Kinase-dead BRAF and oncogenic RAS cooperate to drive tumor progression through CRAF”, Cell 140:209-21, 2010; and Blasco R B, Francoz S, Santamaria D, et al., “c-Raf, but not B-Raf, is essential for development of K-Ras oncogene-driven non-small cell lung carcinoma”, Cancer Cell 19:652-63, 2011. Problematically, 5-20% of patients receiving BRAF^{V600}therapies develop squamous cell carcinomas (SCCs), which is likely driven through the paradoxical activation of the MAPK pathway.

A need therefore exists for improved treatments for cancers having KRAS, NRAS and RAF mutations.

### BRIEF DESCRIPTION

The present disclosure provides a method of treating a subject having cancer characterized by a mutated MAPK signaling pathway. The method comprises: (i) administering to said subject a therapy consisting essentially of (ii) a therapeutically effective amount of a RAF inhibitor and (iii) a therapeutically effective amount of a PD-1 axis inhibitor.

In some aspects, the subject is treated with: (i) a RAF inhibitor in a dose of about 250 mg, about 300 mg, about 350 mg, about 400 mg, about 450 mg, or about 500 mg twice per day and (ii) a PD-1 axis inhibitor in a dose of from about 400 mg to about 1200 mg, from about 600 mg to about 1000 mg, from about 700 mg to about 900 mg, or about 840 mg.

In some particular aspects, the RAF inhibitor is belvarafenib or a pharmaceutically acceptable salt thereof and/or the PD-1 axis inhibitor is a PD-L1 inhibitor. In some aspects, PD-L1 inhibitor is atezolizumab.

As used herein, HM95573 refers to belvarafenib.

### BRIEF DESCRIPTION OF THE DRAWINGS

FIG. 1 is a plot of mouse body weight change versus time for a K1735 syngeneic model for belvarafenib monotherapy (7.5 mg/kg and 15 mg/kg), atezolizumab monotherapy (10 mg/kg), combination therapy at 7.5 mg/kg belvarafenib and 10 mg/kg atezolizumab, and combination therapy at 15 mg/kg belvarafenib and 10 mg/kg atezolizumab of the present disclosure. The vehicle is represented by the top line. Points: mean of relative body weight; bars, S.E.M.

FIG. 2 is a plot of mouse tumor volume versus time for a K1735 syngeneic model for belvarafenib monotherapy (7.5 mg/kg and 15 mg/kg), atezolizumab monotherapy (10 mg/kg), belvarafenib/atezolizumab combination therapy ((i) 7.5 mg/kg and 10 mg/kg and (ii) 15 mg/kg and 10 mg/kg) of the present disclosure. In FIG. 2: * refers to P<0.05; **** refers to P<0.0001; § refers to P<0.05 compared with 10 mg/kg atezolizumab; and # refers to P<0.05 compared with 15 mg/kg belvarafenib. P values were calculated using two-way ANOVA. The vehicle is represented by the top line. Points: mean of tumor volume; bars: S.E.M.

FIG. 3 is a plot of CD3+CD8+ T cells of for a K1735 syngeneic mouse model for belvarafenib monotherapy (15 mg/kg), atezolizumab monotherapy (10 mg/kg), and belvarafenib/atezolizumab combination therapy (15 mg/kg and 10 mg/kg) in K1735 syngeneic mouse model. In FIG. 3: *** refers to P<0.001 compared to vehicle control; ### refers to P<0.001 compared with 15 mg/kg belvarafenib; and § refers to P<0.05 compared with atezolizumab 10 mg/kg. P values were calculated using one-way ANOVA. Point, mean of CD3+CD8+ T cells: bars, S.E.M.

FIG. 4A is a plot of tumor volume (mm³) versus days in a CT26 syngeneic mouse model for KRASG12D CRC for oral treatment with a vehicle where the light lines are results for individual animals in the group and the dark line is the average for the group. FIG. 4B is a plot of tumor volume versus days in the syngeneic mouse model for oral treatment with 5 mg/kg Mu IgG1 (6E11) WT twice a week for three weeks where the light lines are results for individual animals in the group, the dark solid line is the average for the group, and the dark dashed line is the reference fit. FIG. 4C is a plot of tumor volume versus days in the syngeneic mouse model for oral treatment with 10 mg/kg GDC-5573 (belvarafenib) daily for three weeks where the light lines are results for individual animals in the group, the dark solid line is the average for the group, and the dark dashed line is the reference fit. FIG. 4D is a plot of tumor volume versus days in the syngeneic mouse model for the combination of oral treatment with 5 mg/kg Mu IgG1 (6E11) WT twice a week for three weeks and oral treatment with 10 mg/kg GDC-5573 (belvarafenib) daily for three weeks where the light lines are results for individual animals in the group, the dark solid line is the average for the group, and the dark dashed line is the reference fit.

FIG. 5A is a plot of body weight change (%) versus days in a CT26 syngeneic mouse model for KRASG12D CRC for oral treatment with a vehicle where the light lines are results for individual animals in the group and the dark line is the average for the group. FIG. 5B is a plot of body weight change (%) versus days in the syngeneic mouse model for oral treatment with 5 mg/kg Mu IgG1 (6E11) WT twice a week for three weeks where the light lines are results for individual animals in the group and the dark line is the average for the group. FIG. 5C is a plot of body weight change (%) versus days in the syngeneic mouse model for oral treatment with 10 mg/kg GDC-5573 (belvarafenib) daily for three weeks where the light lines are results for individual animals in the group and the dark line is the average for the group. FIG. 5D is a plot of body weight change (%) versus days in the syngeneic mouse model for the combination of oral treatment with 5 mg/kg Mu IgG1 (6E11) WT twice a week for three weeks and oral treatment with 10 mg/kg GDC-5573 (belvarafenib) daily for three weeks where the light lines are results for individual animals in the group and the dark line is the average for the group.

FIG. 6A is an overlay fits tumor volume for FIGS. 4A to 4D. FIG. 6B is an overlay fits body weight change for FIGS. 5A to 5D.

FIG. 7A is a plot of tumor volume (mm3) versus days in an EMT6 syngeneic mouse model for KRASG12D TNBC for oral treatment with a vehicle where the light lines are results for individual animals in the group, and the dark line is the average for the group. FIG. 7B is a plot of tumor volume versus days in the syngeneic mouse model for oral treatment with 5 mg/kg Mu IgG1 (6E11) WT twice a week for three weeks where the light lines are results for individual animals in the group, the dark solid line is the average for the group, and the dark dashed line is the reference fit. FIG. 7C is a plot of tumor volume versus days in the syngeneic mouse model for oral treatment with 10 mg/kg GDC-5573 (belvarafenib) daily for three weeks where the light lines are results for individual animals in the group, the dark solid line is the average for the group, and the dark dashed line is the reference fit. FIG. 7D is a plot of tumor volume versus days in the syngeneic mouse model for the combination of oral treatment with 5 mg/kg Mu IgG1 (6E11) WT twice a week for three weeks and oral treatment with 10 mg/kg GDC-5573 (belvarafenib) daily for three weeks where the light lines are results for individual animals in the group, the dark solid line is the average for the group, and the dark dashed line is the reference fit.

FIG. 8A is a plot of body weight change (%) versus days in an EMT6 syngeneic mouse model for KRASG12D TNBC for oral treatment with a vehicle where the light lines are results for individual animals in the group and the dark line is the average for the group. FIG. 8B is a plot of body weight change (%) versus days in the syngeneic mouse model for oral treatment with 5 mg/kg Mu IgG1 (6E11) WT twice a week for three weeks where the light lines are results for individual animals in the group and the dark line is the average for the group. FIG. 8C is a plot of body weight change (%) versus days in the syngeneic mouse model for oral treatment with 10 mg/kg GDC-5573 (belvarafenib) daily for three weeks where the light lines are results for individual animals in the group and the dark line is the average for the group. FIG. 8D is a plot of body weight change (%) versus days in the syngeneic mouse model for the combination of oral treatment with 5 mg/kg Mu IgG1 (6E11) WT twice a week for three weeks and oral treatment with 10 mg/kg GDC-5573 (belvarafenib) daily for three weeks where the light lines are results for individual animals in the group and the dark line is the average for the group.

FIG. 9A shows overlay fits tumor volume for FIGS. 7A to 7D. FIG. 9B shows overlay fits body weight change for FIGS. 8A to 8D.

### DETAILED DESCRIPTION

The present disclosure is directed to the treatment of cancer characterized by a mutated MAPK signaling pathway with the combination of a RAF inhibitor and a PD-1 axis inhibitor, more particularly to the combination of a RAF inhibitor and a PD-L1 inhibitor, and still more particularly to the combination of belvarafenib and atezolizumab.

### Definitions

As used herein, the term “cancer” refers to or describes the physiological condition in mammals that is typically characterized by unregulated cell growth. A “tumor” comprises one or more cancerous cells.

As used herein, the terms “patient” and “subject” refer to animals such as mammals, including, but not limited to, primates (e.g., humans), cows, sheep, goats, horses, dogs, cats, rabbits, rats, mice and the like. In certain aspects, the patient or subject is a human.

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. For example, an individual is successfully “treated” if one or more symptoms associated with cancer are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, and/or prolonging survival of individuals.

As used herein, the phrase “therapeutically effective amount” refers to an amount of one or more drug compounds that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the overall response rate (ORR). A therapeutically effective amount herein may vary according to factors such as the disease state, age, sex, and weight of the patient, and the ability of the agent to elicit a desired response in the individual. A therapeutically effective amount is also one in which a toxic or detrimental effect of the treatment is outweighed by the therapeutically beneficial effect. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, and enhancing effect of another medication such as via targeting, delaying the progression of the disease, and/or prolonging survival. In the case of a cancer or a tumor, a therapeutically effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. A therapeutically effective amount can be administered in one or more administrations. For purposes of this invention, a therapeutically effective amount of drug, compound, pharmaceutical composition, or pharmaceutical formulation is an amount sufficient to accomplish prophylactic or therapeutic treatment either directly or indirectly. As is understood in the clinical context, a therapeutically effective amount of a drug, compound, or pharmaceutical composition may or may not be achieved in combination with another drug, compound, or pharmaceutical composition. Thus, a therapeutically effective amount may be considered in the context of administering one or more therapeutic agents, and a single agent may be considered to be given in a therapeutically effective amount if, in combination with one or more other agents, a desirable result may be or is achieved.

As used herein, “in combination with” refers to administration of one treatment modality in addition to another treatment modality. As such, “in combination with” refers to administration of one treatment modality before, during, or after administration of the other treatment modality to the individual.

As used herein, the term “pharmaceutical formulation” refers to a preparation which is in such form as to permit the biological activity of the active ingredient to be effective, and which contains no additional components which are unacceptably toxic to a subject to which the formulation would be administered. Such formulations are sterile. “Pharmaceutically acceptable” excipients (vehicles, additives) are those which can reasonably be administered to a subject mammal to provide an effective dose of the active ingredient employed.

As used herein, “C” with reference to maximum, minimum, or other metric, refers to drug concentration in plasma.

As used herein “area under concentration curve” (AUC) refers to the area under a fitted plasma concentration versus time curve. AUC0-∞ refers to area under curve baseline-infinity. AUC0-T is total exposure.

As used herein “inhibit” refers to a decrease in the activity of the target enzyme, as compared to the activity of that enzyme in the absence of the inhibitor. In some aspects, the term “inhibit” means a decrease in activity of at least about 5%, at least about 10%, at least about 20%, at least about 25%, at least about 50%, at least about 60%, at least about 70%, at least about 80%, at least about 90%, or at least about 95%. In other aspects, inhibit means a decrease in activity of about 5% to about 25%, about 25% to about 50%, about 50% to about 75%, or about 75% to 100%. In some aspects, inhibit means a decrease in activity of about 95% to 100%, e.g., a decrease in activity of 95%, 96%, 97%, 98%, 99%, or 100%. Such decreases can be measured using a variety of techniques that would be recognizable by one of skill in the art.

As used herein, “progression free survival” (PFS) refers to the time from the treatment of the disease to the first occurrence of disease progression or relapse.

As used herein, “partial response” (PR) refers to at least a 30% decrease in the sum of diameters of target lesions, taking as reference the baseline sum of diameters.

As used herein, “complete response” (CR) refers to disappearance of all target lesions.

As used herein, “delaying the progression” of a disease means to defer, hinder, slow, retard, stabilize, and/or postpone development of the disease (such as cancer).

This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

As used herein, “progressive disease” (PD) refers to at least a 20% increase in the sum of diameters of target lesions, taking as reference the smallest sum on study (nadir), including baseline and an absolute increase of at least 5 mm. The appearance of one or more new lesions is also considered progression.

As used herein, “overall response rate” (ORR) refers to the rate of a PR or CR occurring after randomization and confirmed ≥28 days later as determined by the investigator using RECIST v1.1.

As used herein, “duration of response” (DOR) refers to the time from the first occurrence of a documented objective response to the time of relapse, as determined by the investigator using RECIST v1.1 or death from any cause during the study, whichever occurs first.

As used herein, the term “RAF inhibitor(s)” refers to a molecule that inhibits at least one of three subtypes (A-RAF, B-RAF, C-RAF) in the MAPK signaling pathway downstream of RAS.

As used herein, the term “MAPK” refers to the mitogen-activated protein kinase pathway or signaling pathway. Also termed the Ras-Raf-MEK-ERK pathway, the MAPK pathway, is a chain or pathway of proteins in the cell that communicates a signal from a receptor on the surface of the cell to the DNA in the nucleus of the cell. In the MAPK pathway, activated RAS activates the protein kinase activity of RAF kinase, RAF kinase phosphorylates and activates MEK (MEK1 and MEK2), MEK phosphorylates and activates a mitogen-activated protein kinase (MAPK) ERK1 and ERK2 (MAPK3 and MAPK1). MAPK phosphorylates ribosomal protein S6 kinase (RPS6KA1; RSK).

As used herein, the term “PD-1 axis inhibitor” refers to a molecule that inhibits the interaction of a PD-1 axis binding partner with either one or more of its binding partner, so as to remove T-cell dysfunction resulting from signaling on the PD-1 signaling axis—with a result being to restore or enhance T-cell function (e.g., proliferation, cytokine production, target cell killing). As used herein, a PD-1 axis inhibitor includes a PD-1 inhibitor, a PD-L 1 inhibitor, and a PD-L2 inhibitor.

As used herein, the term “PD-1 inhibitor” refers to a molecule that decreases, blocks, inhibits, abrogates or interferes with signal transduction resulting from the interaction of PD-1 with one or more of its binding partners, such as PD-L1 and PD-L2. In some embodiments, the PD-1 inhibitor is a molecule that inhibits the binding of PD-1 to one or more of its binding partners. In a specific aspect, the PD-1 inhibitor inhibits the binding of PD-1 to PD-L1 and/or PD-L2. For example, PD-1 inhibitors include anti-PD-1 antibodies, antigen binding fragments thereof, immunoadhesins, fusion proteins, oligopeptides and other molecules that decrease, block, inhibit, abrogate or interfere with signal transduction resulting from the interaction of PD-1 with PD-L1 and/or PD-L2. In one embodiment, a PD-1 inhibitor reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some embodiments, the PD-1 inhibitor is an anti-PD-1 antibody.

As used herein, the term “PD-L1 inhibitor” refers to a molecule that decreases, blocks, inhibits, abrogates or interferes with signal transduction resulting from the interaction of PD-L1 with either one or more of its binding partners, such as PD-1, B7-1. In some embodiments, a PD-L1 inhibitor is a molecule that inhibits the binding of PD-L1 to its binding partners. In a specific aspect, the PD-L1 inhibitor inhibits binding of PD-L1 to PD-1 and/or B7-1. In some embodiments, the PD-L1 inhibitor include anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1, B7-1. In one embodiment, a PD-L1 inhibitor reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some embodiments, a PD-L1 inhibitor is an anti-PD-L1 antibody.

As used herein, the term “PD-L2 inhibitor” refers to a molecule that decreases, blocks, inhibits, abrogates or interferes with signal transduction resulting from the interaction of PD-L2 with either one or more of its binding partners, such as PD-1. In some embodiments, a PD-L2 inhibitor is a molecule that inhibits the binding of PD-L2 to one or more of its binding partners. In a specific aspect, the PD-L2 inhibitor inhibits binding of PD-L2 to PD-1. In some embodiments, the PD-L2 inhibitor include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one embodiment, a PD-L2 inhibitor reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some embodiments, a PD-L2 inhibitor is an immunoadhesin.

The term “pharmaceutically acceptable salts” denotes salts which are not biologically or otherwise undesirable. Pharmaceutically acceptable salts include both acid and base addition salts. The phrase “pharmaceutically acceptable” indicates that the substance or composition is compatible chemically and/or toxicologically, with the other ingredients comprising a formulation, and/or the mammal being treated therewith. Acid addition salts are formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, malonic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid “mesylate”, ethanesulfonic acid, p-toluenesulfonic acid, and salicyclic acid. Base addition salts are formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, and polyamine resins.

The term “antibody” herein is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

An “isolated” antibody is one which has been identified and separated and/or recovered from a component of its natural environment. Contaminant components of its natural environment are materials which would interfere with research, diagnostic or therapeutic uses for the antibody, and may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. Isolated antibody includes the antibody in situ within recombinant cells since at least one component of the antibody's natural environment will not be present. Ordinarily, however, isolated antibody will be prepared by at least one purification step.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The term “constant domain” refers to the portion of an immunoglobulin molecule having a more conserved amino acid sequence relative to the other portion of the immunoglobulin, the variable domain, which contains the antigen binding site. The constant domain contains the CH1, CH2 and CH3 domains (collectively, CH) of the heavy chain and the CHL (or CL) domain of the light chain.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The “light chains” of antibodies (immunoglobulins) from any mammalian species can be assigned to one of two clearly distinct types, called kappa (“κ”) and lambda (“λ”), based on the amino acid sequences of their constant domains.

The term IgG “isotype” or “subclass” as used herein is meant any of the subclasses of immunoglobulins defined by the chemical and antigenic characteristics of their constant regions.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called a, y, c, y, and p, respectively. The subunit structures and three-dimensional configurations of different classes of immunoglobulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

The terms “full length antibody,” “intact antibody” and “whole antibody” are used herein interchangeably to refer to an antibody in its substantially intact form, not antibody fragments as defined below. The terms particularly refer to an antibody with heavy chains that contain an Fc region.

“Antibody fragments” comprise a portion of an intact antibody, preferably comprising the antigen binding region thereof. In some embodiments, the antibody fragment described herein is an antigen-binding fragment. Examples of antibody fragments include Fab, Fab′, F(ab′)2, and Fv fragments; diabodies; linear antibodies; single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three HVRs of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv, see, e.g., Pluckthun, in The Pharmacology of Monoclonal Antibodies, vol. 113, Rosenburg and Moore eds., (Springer-Verlag, New York, 1994), pp. 269-315.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al., Proc. Natl. Acad. Sci. USA 81:6851-6855 (1984)). Chimeric antibodies include PRIMATTZED® antibodies wherein the antigen-binding region of the antibody is derived from an antibody produced by, e.g., immunizing macaque monkeys with the antigen of interest.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a HVR of the recipient are replaced by residues from a HVR of a non-human species (donor antibody) such as mouse, rat, rabbit, or nonhuman primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

A “species-dependent antibody” is one which has a stronger binding affinity for an antigen from a first mammalian species than it has for a homologue of that antigen from a second mammalian species. Normally, the species-dependent antibody “binds specifically” to a human antigen (e.g., has a binding affinity (Kd) value of no more than about 1×10-7 M, preferably no more than about 1×10-8 M and preferably no more than about 1×10-9 M) but has a binding affinity for a homologue of the antigen from a second nonhuman mammalian species which is at least about 50 fold, or at least about 500 fold, or at least about 1000 fold, weaker than its binding affinity for the human antigen. The species-dependent antibody can be any of the various types of antibodies as defined above, but preferably is a humanized or human antibody.

The term “hypervariable region,” “HVR,” or “HV,” when used herein refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B (Kabat |
| | | | | Numbering) |
| H1 | H31-H35 | H26-H35 | H26-H32 | H30-H35 (Chothia |
| | | | | Numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

“Framework” or “FR” residues are those variable domain residues other than the HVR residues as herein defined.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody.

The expression “linear antibodies” refers to the antibodies described in Zapata et al. (1995 Protein Eng, 8(10):1057-1062). Briefly, these antibodies comprise a pair of tandem Fd segments (VH-CH1-VH-CH1) which, together with complementary light chain polypeptides, form a pair of antigen binding regions. Linear antibodies can be bispecific or monospecific.

As use herein, the term “binds”, “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of ≤1 M, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding.

The term “detection” includes any means of detecting, including direct and indirect detection.

### Therapeutic Agents

The present disclosure uses the combination of a RAF inhibitor and a PD-1 axis inhibitor to treat cancer characterized by a mutated MAPK signaling pathway in a subject. In some aspects, (i) the RAF inhibitor is belvarafenib or a pharmaceutically acceptable salt thereof and (ii) the PD-1 axis inhibitor is a PD-L1 inhibitor, and more particularly the PD-L1 inhibitor is atezolizumab (brand name TECENTRIQ®).

The presently disclosed compounds may be administered in any suitable manner known in the art. In some aspects, the compounds may be administered intravenously, intramuscularly, subcutaneously, topically, orally, transdermally, intraperitoneally, intraorbitally, by implantation, by inhalation, intrathecally, intraventricularly, intratumorally, or intranasally.

It is understood that appropriate doses of the active compound depends upon a number of factors within the knowledge of the ordinarily skilled physician. The dose(s) of the active compound will vary, for example, depending upon the age, body weight, general health, gender, and diet of the subject, the time of administration, the route of administration, the rate of excretion, and any drug combination.

It will also be appreciated that the effective dosage of the compound of the present disclosure, or a pharmaceutically acceptable salts, prodrugs, metabolites, or derivatives thereof used for treatment may increase or decrease over the course of a particular treatment. Changes in dosage may result and become apparent from the results of diagnostic assays.

### RAF Inhibitors

Examples of RAF inhibitors within the scope of the present disclosure include belvarafenib, vemurafenib, dabrafenib, encorafenib.

Belvarafenib is disclosed in PCT application WO 2013/100632, has the chemical name 4-amino-N-(1-((3-chloro-2-fluorophenyl)amino)-6-methylisoquinolin-5-yl)thieno[3,2-d]pyrimidine-7-carboxamide (referred to herein as Formula (I)), and has the following chemical structure:

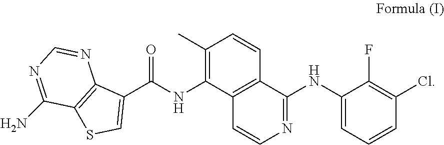 [SMILES: Cc1ccc2c(Nc3cccc(Cl)c3F)nccc2c1NC(=O)c1csc2c(N)ncnc12]

Belvarafenib is well-tolerated and has been discovered to be effective for treatment of certain brain cancers in a subject. A subject within the scope of the disclosure is a mammal, including, but not limited to, a human or non-human mammal, such as a bovine, equine, canine, sheep or feline. In some aspects, the subject is a human.

Belvarafenib is a highly potent and selective type II RAF dimer inhibitor (a pan-RAF inhibitor) that provides for selective inhibition of BRAF and CRAF isoforms. In contrast with BRAFV600-selective monomer inhibitors, belvarafenib does not activate the MAPK pathway in non-BRAF V600 mutant cells, but instead sustains the suppression of MAPK signaling by inhibiting BRAF and CRAF dimers, and results in reduced cell proliferation and increased antitumor activity in both BRAFV600- and RAS-mutant tumors.

Belvarafenib inhibits phosphorylation of MEK and ERK in the MAPK pathway in BRAF- or RAS-mutant melanoma, NSCLC, and CRC cell lines. Belvarafenib has been demonstrated to inhibit the growth of BRAF- or RAS-mutant melanoma, NSCLC, CRC, and thyroid cancer cell lines in vitro.

Belvarafenib is a potent and selective inhibitor of the RAF kinases including BRAF V600E mutant (IC50=7 nM), BRAF wild type (IC50=41 nM), and RAF-1 (CRAF) (IC50=2 nM) in vitro. When tested in a panel of 189 kinase assays, belvarafenib showed inhibitory activity against 7 other receptor tyrosine kinases (RTKs) (colony stimulating factor 1 receptor (CSF1R), formerly McDonough feline sarcoma (FMS) homolog, discoidin domain receptor tyrosine kinase 1 (DDR1), discoidin domain receptor tyrosine kinase 2 (DDR2), EPHA2, EPHA7, EPHA8, and EPHB2) with >90% inhibition at 1 μM.

The in vitro antitumor effects of belvarafenib have translated into efficacy in various mouse xenograft models. Belvarafenib shows dose-dependent inhibition of tumor growth in mouse xenograft models as a monotherapy against BRAF- and NRAS-mutant melanoma, against KRAS mutant non-small cell lung cancer (NSCLC), and against BRAF mutant colorectal cancer (CRC) mouse xenograft models.

Belvarafenib has been shown in clinical trials to provide safe and efficacious therapy against a number of cancers.

For instance, a completed, open-label, Phase Ia, dose-escalation investigated several doses and schedules of belvarafenib in patients with solid tumors harboring mutations in BRAF, KRAS, or NRAS genes. Efficacy was analyzed for 67 of 72 subjects who had at least 1 post-baseline tumor assessment. Best overall response rate (BORR) was 8.96% (6/67 subjects), objective response rate (ORR) was 4.48% (3/67 subjects) with partial response (PR) as confirmed best overall response (2 subjects with melanoma and a subject with gastrointestinal stromal tumor). Disease control was observed in 50.57% (34/67) of subjects treated with belvarafenib 100 mg QD dose level or above. Fifty-nine (88.06%) subjects developed an event (progression of disease or death), all of which were reported as progressive disease (PD). In addition, median progression-free survival was 11.53 weeks and the 95% confidence interval for the median was [7.12 weeks, 13.38 weeks). In updated results, BORR was 10.45% (7/67 subjects) and the 95% exact confidence interval was [4.30%, 20.35%]; ORR remains at 4.48% (3/67 subjects). In addition, subgroup re-analysis for BRAF-mutant melanoma subjects showed 7.69% (1/13 subjects) of BORR, DCR, median PFS, and time to progression were not changed in total subjects. Median DOR was elevated to 30.18 weeks in 800 mg BID group and 23.99 weeks in total group including DOR of a subject which is 100.29 weeks.

In another open-label, Phase Ib, dose-expansion study, belvarafenib was evaluated at a dose of 450 mg BID in patients with solid tumors harboring mutations in BRAF, KRAS, or NRAS genes. Efficacy was analyzed for 59 of 63 subjects who had at least 1 dose of belvarafenib after enrollment and had at least 1 post-baseline tumor assessment. BORR was 11.86% (7/59 subjects), ORR was 6.78% (4/59 subjects) with PR as confirmed best overall response (3 subjects with melanoma and a subject with CRC). Disease control was observed in 35.59% (21/59) of subjects. Fifty (84.75%) of 59 subjects developed an event (progression of disease or death), all of which were reported as PD except 1 death case. In addition, median progression-free survival (PFS) was 7.83 weeks and the 95% confidence interval for the median was [7.26 weeks, 8.26 weeks]. Median duration of response (DOR) of a total response in this study was 15.66 weeks from 7 responders. Among them, 2 BRAF-mutant melanoma responders showed 22.49 weeks of median DOR.

In another Phase I, single dose, randomized, crossover relative bioavailability and food effect study in healthy subjects, the influence of a formulation change from the Phase I to Phase II tablet on belvarafenib exposure was evaluated. A total of 18 healthy subjects were enrolled in the study and received the following randomized treatments: one 150-mg and one 50-mg Phase I tablet in a fed state, two 100-mg Phase II tablets in a fed state, or two 100-mg Phase II tablets in a fasted state, with a 18-day washout between treatments. There was a positive effect of food on belvarafenib exposure in the fed state compared to the fasted state. Belvarafenib exposure, Cmax and AUC0-inf, were increased by approximately 2.2- and 2.8-fold, respectively, when belvarafenib was administered in the fed state compared to the fasted state in healthy subjects at a 200 mg single dose. No serious adverse events, adverse events of special interest, or deaths were reported in the study.

Belvarafenib, or a pharmaceutically acceptable salt thereof, is suitably dosed at about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, or about 1500 mg per day, and any range constructed therefrom, where the dosage is based on active ingredient. Suitable belvarafenib daily dose ranges may be from about 100 mg to about 1500 mg, from about 250 mg to about 1250 mg, from about 500 mg to about 1000 mg, or from about 700 mg to about 900 mg. In some aspects, the subject is treated twice per day with belvarafenib or a pharmaceutically acceptable salt thereof in order to achieve a total daily dose. In some such aspects, the subject is treated with about 250 mg, about 300 mg, about 350 mg, about 400 mg, about 450 mg, or about 500 mg of belvarafenib or a pharmaceutically acceptable salt thereof twice per day. Other dosing regimens may be used to achieve a total daily dose, such as three doses per day or four doses per day. In any such dosing regimen, such as two, three or four times per day, each dose may suitably be about equal. For instance, if the daily dose is 900 mg, two daily doses of 450 mg each or three daily doses of 300 mg each could be used.

In some aspects, belvarafenib may be dosed on days 1 to 21 of a 28-day cycle. In some aspects, belvarafenib may be dosed on days 1 to 28 of a 28-day cycle.

### PD-1 Axis Inhibitors

In accordance with the present disclosure, a PD-1 axis inhibitor may more particularly refer to a PD-1 inhibitor, a PD-L1 inhibitor, or a PD-L2 inhibitor. Alternative names for “PD-1” include CD279 and SLEB2. Alternative names for “PD-L1” include B7-H1, B7-4, CD274, and B7-H. Alternative names for “PD-L2” include B7-DC, Btdc, and CD273. In some embodiments, PD-1, PD-L1, and PD-L2 are human PD-1, PD-L1 and PD-L2.

In some embodiments, the PD-1 inhibitor is a molecule that inhibits the binding of PD-1 to its ligand binding partners. In a specific aspect the PD-1 ligand binding partners are PD-L1 and/or PD-L2. In another embodiment, a PD-L1 inhibitor is a molecule that inhibits the binding of PD-L1 to its binding partners. In a specific aspect, PD-L1 binding partners are PD-1 and/or B7-1. In another embodiment, the PD-L2 inhibitor is a molecule that inhibits the binding of PD-L2 to its binding partners. In a specific aspect, a PD-L2 binding partner is PD-1. The inhibitor may be an antibody, an antigen binding fragment thereof, an immunoadhesin, a fusion protein, or oligopeptide.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, lambrolizumab, and CT-011. In some embodiments, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PD-1 inhibitor is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342.

In some embodiments, the anti-PD-1 antibody is nivolumab (CAS Registry Number: 946414-94-4). In a still further embodiment, provided is an isolated anti-PD-1 antibody comprising a heavy chain variable region comprising the heavy chain variable region amino acid sequence from SEQ ID NO:1 and/or a light chain variable region comprising the light chain variable region amino acid sequence from SEQ ID NO:2. In a still further embodiment, provided is an isolated anti-PD-1 antibody comprising a heavy chain and/or a light chain sequence, wherein:
(a) the heavy chain sequence has at least 85%, at least 90%, at least 91%, at least 92%, at least 93%, at least 94%, at least 95%, at least 96%, at least 97%, at least 98%, at least 99% or 100% sequence identity to the heavy chain sequence: QVQLVESGGGVVQPGRSLRLDCKASGITFSNSGMHWVRQAPGKGLEWVA VIWYDGSKRYYADSVKGRFTISRDNSKNTLFLQMNSLRAEDTAVYYCATN DDYWGQGTLVTVSSASTKGPSVFPLAPCSRSTSESTAALGCLVKDYFPEPV TVSWNSGALTSGVHTFPAVLQSSGLYSLSSVVTVPSSSLGTKTYTCNVDHK PSNTKVDKRVESKYGPPCPPCPAPEFLGGPSVFLFPPKPKDTLMISRTPEVTC VVVDVSQEDPEVQFNWYVDGVEVHNAKTKPREEQFNSTYRVVSVLTVLH QDWLNGKEYKCKVSNKGLPSSIEKTISKAKGQPREPQVYTLPPSQEEMTKN QVSLTCLVKGFYPSDIAVEWESNGQPENNYKTTPPVLDSDGSFFLYSRLTV DKSRWQEGNVFSCSVMHEALHNHYTQKSLSLSLGK (SEQ ID NO:1), or
(b) the light chain sequences has at least 85%, at least 90%, at least 91%, at least 92%, at least 93%, at least 94%, at least 95%, at least 96%, at least 97%, at least 98%, at least 99% or 100% sequence identity to the light chain sequence:

(SEQ ID NO: 2)
EIVLTQSPATLSLSPGERATLSCRASQSVSSYLAWYQQKPGQAPR
LLIYDASNRATGIPARFSGSGSGTDFTLTISSLEPEDFAVYYCQQ
SSNWPRTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC.

In some embodiments, the anti-PD-1 antibody is pembrolizumab (CAS Registry Number: 1374853-91-4). In a still further embodiment, provided is an isolated anti-PD-1 antibody comprising a heavy chain variable region comprising the heavy chain variable region amino acid sequence from SEQ ID NO:3 and/or a light chain variable region comprising the light chain variable region amino acid sequence from SEQ ID NO:4. In a still further embodiment, provided is an isolated anti-PD-1 antibody comprising a heavy chain and/or a light chain sequence, wherein:
(a) the heavy chain sequence has at least 85%, at least 90%, at least 91%, at least 92%, at least 93%, at least 94%, at least 95%, at least 96%, at least 97%, at least 98%, at least 99% or 100% sequence identity to the heavy chain sequence: QVQLVQSGVE VKKPGASVKV SCKASGYTFT NYYMYWVRQA PGQGLEWMGG INPSNGGTNF NEKFKNRVTL TTDSSTTTAY MELKSLQFDD TAVYYCARRDYRFDMGFDYW GQGTTVTVSS ASTKGPSVFP LAPCSRSTSE STAALGCLVKDYFPEPVTVS WNSGALTSGV HTFPAVLQSS GLYSLSSVVT VPSSSLGTKTYTCNVDHKPS NTKVDKRVES KYGPPCPPCP APEFLGGPSV FLFPPKPKDTLMISRTPEVT CVVVDVSQED PEVQFNWYVD GVEVHNAKTK PREEQFNSTYRVVSVLTVLH QDWLNGKEYK CKVSNKGLPS SIEKTISKAK GQPREPQVYTLPPSQEEMTK NQVSLTCLVK GFYPSDIAVE WESNGQPENN YKTTPPVLDSDGSFFLYSRL TVDKSRWQEG NVFSCSVMHE ALHNHYTQKS LSLSLGK (SEQ ID NO:3), or
(b) the light chain sequences have at least 85%, at least 90%, at least 91%, at least 92%, at least 93%, at least 94%, at least 95%, at least 96%, at least 97%, at least 98%, at least 99% or 100% sequence identity to the light chain sequence:

(SEQ ID NO: 4)
EIVLTQSPATLSLSPGERATLSCRASKGVSTSGYSYLHWYQQKPG
QAPRLLIYLASYLESGVPARFSGSGSGTDFTLTISSLEPEDFAVY
YCQHSRDLPLTFGGGTKVEIKRTVAAPSVFIFPPSDEQLKSGTAS
VVCLLNNFYPREAKVQWKVDNALQSGNSQESVTEQDSKDSTYSLS
STLTLSKADYEKHKVYACEVTHQGLSSPVTKSFNRGEC.

In some embodiments, the PD-L1 inhibitor is anti-PD-L1 antibody. In some embodiments, the anti-PD-L1 inhibitor is selected from the group consisting of YW243.55.S70, MPDL3280A (atezolizumab), MDX-1105, and MEDI4736. MDX-1105, also known as BMS-936559, is an anti-PD-L1 antibody described in WO2007/005874. Antibody YW243.55.S70 (heavy and light chain variable region sequences shown in SEQ ID Nos. 5 and 6, respectively) is an anti-PD-L1 described in WO 2010/077634 A1. MEDI4736 is an anti-PD-L1 antibody described in WO2011/066389 and US2013/034559.

Examples of anti-PD-L1 antibodies useful for the methods of this invention, and methods for making thereof are described in PCT patent application WO 2010/077634 A1 and U.S. Pat. No. 8,217,149, which are incorporated herein by reference.

In some embodiments, the PD-1 axis inhibitor is an anti-PD-L1 antibody. In some embodiments, the anti-PD-L1 antibody is capable of inhibiting binding between PD-L1 and PD-1 and/or between PD-L1 and B7-1. In some embodiments, the anti-PD-L1 antibody is a monoclonal antibody. In some embodiments, the anti-PD-L1 antibody is an antibody fragment selected from the group consisting of Fab, Fab′-SH, Fv, scFv, and (Fab′)2 fragments. In some embodiments, the anti-PD-L1 antibody is a humanized antibody. In some embodiments, the anti-PD-L1 antibody is a human antibody.

The anti-PD-L1 antibodies useful in this invention, including compositions containing such antibodies, such as those described in WO 2010/077634 A1. In some embodiments, the anti-PD-L1 antibody comprises a heavy chain variable region comprising the amino acid sequence of SEQ ID NO:7 or 8 (Infra) and a light chain variable region comprising the amino acid sequence of SEQ ID NO:9 (Infra).

In one embodiment, the anti-PD-L1 antibody contains a heavy chain variable region polypeptide comprising an HVR-H1, HVR-H2 and HVR-H3 sequence, wherein:
(a) the HVR-H1 sequence is GFTFSX₁SWIH (SEQ ID NO:10);
(b) the HVR-H2 sequence is AWIX₂PYGGSX₃YYADSVKG (SEQ ID NO:11);
(c) the HVR-H3 sequence is RHWPGGFDY (SEQ ID NO:12);
further wherein: X₁ is D or G; X₂ is S or L; X₃ is T or S.

In one specific aspect, X1 is D; X₂ is S and X₃ is T. In another aspect, the polypeptide further comprises variable region heavy chain framework sequences juxtaposed between the HVRs according to the formula: (HC-FR1)-(HVR-H1)-(HC-FR2)-(HVR-H2)-(HC-FR3)-(HVR-H3)-(HC-FR4). In yet another aspect, the framework sequences are derived from human consensus framework sequences. In a further aspect, the framework sequences are VH subgroup III consensus framework. In a still further aspect, at least one of the framework sequences is the following:

HC-FR1 is
(SEQ ID NO: 13)
EVQLVESGGGLVQPGGSLRLSCAAS
HC-FR2 is
(SEQ ID NO: 14)
WVRQAPGKGLEWV
HC-FR3 is
(SEQ ID NO: 15)
RFTISADTSKNTAYLQMNSLRAEDTAVYYCAR
HC-FR4 is
(SEQ ID NO: 16)
WGQGTLVTVSA.

In a still further aspect, the heavy chain polypeptide is further combined with a variable region light chain comprising an HVR-L1, HVR-L2 and HVR-L3, wherein:

(a) the HVR-L1 sequence is
(SEQ ID NO: 17)
RASQX₄X₅X₆TX₇X₈A;
(b) the HVR-L2 sequence is
(SEQ ID NO: 18)
SASX₉LX₁₀S;
(c) the HVR-L3 sequence is
(SEQ ID NO: 19)
QQX₁₁X₁₂X₁₃X₁₄PX₁₅T;

further wherein: X₄ is D or V; X₅ is V or I; X₆ is S or N; X₇ is A or F; X_{g} is V or L; X₉ is F or T; X₁₀ is Y or A; Xₙ is Y, G, F, or S; X₁₂ is L, Y, F or W; X₁₃ is Y, N, A, T, G, F or I; X₁₄ is H, V, P, T or I; X₁₅ is A, W, R, P or T.

In a still further aspect, X₄ is D; X₅ is V; X₆ is S; X₇ is A; X₈ is V; X₉ is F; X₁₀ is Y; X₁₁ is Y; X₁₂ is L; X₁₃ is Y; X₁₄ is H; X₁₅ is A. In a still further aspect, the light chain further comprises variable region light chain framework sequences juxtaposed between the HVRs according to the formula: (LC-FR1)-(HVR-L1)-(LC-FR2)-(HVR-L2)-(LC-FR3)-(HVR-L3)-(LC-FR4). In a still further aspect, the framework sequences are derived from human consensus framework sequences. In a still further aspect, the framework sequences are VL kappa I consensus framework. In a still further aspect, at least one of the framework sequence is the following:

LC-FR1 is
(SEQ ID NO: 20)
DIQMTQSPSSLSASVGDRVTITC
LC-FR2 is
(SEQ ID NO: 21)
WYQQKPGKAPKLLIY
LC-FR3 is
(SEQ ID NO: 22)
GVPSRFSGSGSGTDFTLTISSLQPEDFATYYC
LC-FR4 is
(SEQ ID NO: 23)
FGQGTKVEIKR.

In another embodiment, provided is an isolated anti-PD-L1 antibody or antigen binding fragment comprising a heavy chain and a light chain variable region sequence, wherein:

the heavy chain comprises and HVR-H1, HVR-H2 and HVR-H3, wherein further:
(i) the HVR-H1 sequence is GFTFSX₁SWIH; (SEQ ID NO:10)
(ii) the HVR-H2 sequence is AWIX₂PYGGSX₃YYADSVKG (SEQ ID NO:11)
(iii) the HVR-H3 sequence is RHWPGGFDY, and (SEQ ID NO:12)
   the light chain comprises and HVR-L1, HVR-L2 and HVR-L3, wherein further:
(i) the HVR-L1 sequence is RASQX₄X5X₆TX₇XgA (SEQ ID NO:17)
(ii) the HVR-L2 sequence is SASX₉LX₁₀S; and (SEQ ID NO:18)
(iii) the HVR-L3 sequence is QQX₁₁X₁₂X₁₃X₁₄PX₁₅T; (SEQ ID NO:19)

Further wherein: X₁ is D or G; X2 is S or L; X3 is T or S; X4 is D or V; X5 is V or I; X6 is S or N; X7 is A or F; X8 is V or L; X9 is F or T; X10 is Y or A; X11 is Y, G, F, or S; X12 is L, Y, F or W; X13 is Y, N, A, T, G, F or I; X14 is H, V, P, T or I; X15 is A, W, R, P or T.

In a specific aspect, X1 is D; X2 is S and X3 is T. In another aspect, X4 is D; X5 is V; X6 is S; X7 is A; X8 is V; X9 is F; X10 is Y; X11 is Y; X12 is L; X13 is Y; X14 is H; X15 is A. In yet another aspect, X1 is D; X2 is S and X3 is T, X4 is D; X5 is V; X6 is S; X7 is A; X8 is V; X9 is F; X10 is Y; X11 is Y; X12 is L; X13 is Y; X14 is H and X15 is A.

In a further aspect, the heavy chain variable region comprises one or more framework sequences juxtaposed between the HVRs as: (HC-FR1)-(HVR-H1)-(HC-FR2)-(HVR-H2)-(HC-FR3)-(HVR-H3)-(HC-FR4), and the light chain variable regions comprises one or more framework sequences juxtaposed between the HVRs as: (LC-FR1)-(HVR-L1)-(LC-FR2)-(HVR-L2)-(LC-FR3)-(HVR-L3)-(LC-FR4). In a still further aspect, the framework sequences are derived from human consensus framework sequences. In a still further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences is the following:

HC-FR1
(SEQ ID NO: 13)
EVQLVESGGGLVQPGGSLRLSCAAS
HC-FR2
(SEQ ID NO: 14)
WVRQAPGKGLEWV
HC-FR3
(SEQ ID NO: 15)
RFTISADTSKNTAYLQMNSLRAEDTAVYYCAR
HC-FR4
(SEQ ID NO: 16)
WGQGTLVTVSA.

In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences is the following:

LC-FR1
SEQ ID NO: 20)
DIQMTQSPSSLSASVGDRVTITC
LC-FR2
(SEQ ID NO: 21)
WYQQKPGKAPKLLIY
LC-FR3
(SEQ ID NO: 22)
GVPSRFSGSGSGTDFTLTISSLQPEDFATYYC
LC-FR4
(SEQ ID NO: 23)
FGQGTKVEIKR.

In a still further specific aspect, the antibody further comprises a human or murine constant region. In a still further aspect, the human constant region is selected from the group consisting of IgG1, IgG2, IgG2, IgG3, IgG4. In a still further specific aspect, the human constant region is IgG1. In a still further aspect, the murine constant region is selected from the group consisting of IgG1, IgG2A, IgG2B, IgG3. In a still further aspect, the murine constant region if IgG2A. In a still further specific aspect, the antibody has reduced or minimal effector function. In a still further specific aspect the minimal effector function results from an “effector-less Fc mutation” or aglycosylation. In still a further embodiment, the effector-less Fc mutation is an N297A or D265A/N297A substitution in the constant region.

In yet another embodiment, provided is an anti-PD-L1 antibody comprising a heavy chain and a light chain variable region sequence, wherein:
(a) the heavy chain further comprises and HVR-H1, HVR-H2 and an HVR-H3 sequence having at least 85% sequence identity to GFTFSDSWIH (SEQ ID NO:24), AWISPYGGSTYYADSVKG (SEQ ID NO:25) and RHWPGGFDY (SEQ ID NO:12), respectively, or
(b) the light chain further comprises an HVR-L1, HVR-L2 and an HVR-L3 sequence having at least 85% sequence identity to RASQDVSTAVA (SEQ ID NO:26), SASFLYS (SEQ ID NO:27) and QQYLYHPAT (SEQ ID NO:28), respectively.

In a specific aspect, the sequence identity is 86%, 87%, 88%, 89%, 90%, 91%, 92%, 93%, 94%, 95%, 96%, 97%, 98%, 99% or 100%. In another aspect, the heavy chain variable region comprises one or more framework sequences juxtaposed between the HVRs as: (HC-FR1)-(HVR-H1)-(HC-FR2)-(HVR-H2)-(HC-FR3)-(HVR-H3)-(HC-FR4), and the light chain variable regions comprises one or more framework sequences juxtaposed between the HVRs as: (LC-FR1)-(HVR-L1)-(LC-FR2)-(HVR-L2)-(LC-FR3)-(HVR-L3)-(LC-FR4). In yet another aspect, the framework sequences are derived from human consensus framework sequences. In a still further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences is the following:

HC-FR1
(SEQ ID NO: 13)
EVQLVESGGGLVQPGGSLRLSCAAS
HC-FR2
(SEQ ID NO: 14)
WVRQAPGKGLEWV
HC-FR3
(SEQ ID NO: 15)
RFTISADTSKNTAYLQMNSLRAEDTAVYYCAR
HC-FR4
(SEQ ID NO: 16)
WGQGTLVTVSA.

In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences is the following:

LC-FR1
(SEQ ID NO: 20)
DIQMTQSPSSLSASVGDRVTITC
LC-FR2
(SEQ ID NO: 21)
WYQQKPGKAPKLLIY
LC-FR3
(SEQ ID NO: 22)
GVPSRFSGSGSGTDFTLTISSLQPEDFATYYC
LC-FR4
(SEQ ID NO: 23)
FGQGTKVEIKR.

In a still further specific aspect, the antibody further comprises a human or murine constant region. In a still further aspect, the human constant region is selected from the group consisting of IgG1, IgG2, IgG2, IgG3, IgG4. In a still further specific aspect, the human constant region is IgG1. In a still further aspect, the murine constant region is selected from the group consisting of IgG1, IgG2A, IgG2B, IgG3. In a still further aspect, the murine constant region if IgG2A. In a still further specific aspect, the antibody has reduced or minimal effector function. In a still further specific aspect the minimal effector function results from an “effector-less Fc mutation” or aglycosylation. In still a further embodiment, the effector-less Fc mutation is an N297A or D265A/N297A substitution in the constant region.

In a still further embodiment, provided is an isolated anti-PD-L1 antibody comprising a heavy chain and a light chain variable region sequence, wherein:
(a) the heavy chain sequence has at least 85% sequence identity to the heavy chain sequence:

(SEQ ID NO: 29)
EVQLVESGGGLVQPGGSLRLSCAASGFTFSDSWIHWVRQAPG
KGLEWVAWISPYGGSTYYADSVKGRFTISADTSKNTAYLQMN
SLRAEDTAVYYCARRHWPGGFDYWGQGTLVTVSA,

or
(b) the light chain sequence has at least 85% sequence identity to the light chain sequence:

(SEQ ID NO: 9)
DIQMTQSPSSLSASVGDRVTITCRASQDVSTAVAWYQQKPG
KAPKLLIYSASFLYSGVPSRFSGSGSGTDFTLTISSLQPED
FATYYCQQYLYHPATFGQGTKVEIKR.

In a specific aspect, the sequence identity is 86%, 87%, 88%, 89%, 90%, 91%, 92%, 93%, 94%, 95%, 96%, 97%, 98%, 99% or 100%. In another aspect, the heavy chain variable region comprises one or more framework sequences juxtaposed between the HVRs as: (HC-FR1)-(HVR-H1)-(HC-FR2)-(HVR-H2)-(HC-FR3)-(HVR-H3)-(HC-FR4), and the light chain variable regions comprises one or more framework sequences juxtaposed between the HVRs as: (LC-FR1)-(HVR-L1)-(LC-FR2)-(HVR-L2)-(LC-FR3)-(HVR-L3)-(LC-FR4). In yet another aspect, the framework sequences are derived from human consensus framework sequences. In a further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences is the following:

HC-FR1
(SEQ ID NO: 13)
EVQLVESGGGLVQPGGSLRLSCAAS
HC-FR2
(SEQ ID NO: 14)
WVRQAPGKGLEWV
HC-FR3
(SEQ ID NO: 15)
RFTISADTSKNTAYLQMNSLRAEDTAVYYCAR
HC-FR4
(SEQ ID NO: 16)
WGQGTLVTVSA.

In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences is the following:

LC-FR1
(SEQ ID NO: 20)
DIQMTQSPSSLSASVGDRVTITC
LC-FR2
(SEQ ID NO: 21)
WYQQKPGKAPKLLIY
LC-FR3
(SEQ ID NO: 22)
GVPSRFSGSGSGTDFTLTISSLQPEDFATYYC
LC-FR4
(SEQ ID NO: 23)
FGQGTKVEIKR.

In a still further specific aspect, the antibody further comprises a human or murine constant region. In a still further aspect, the human constant region is selected from the group consisting of IgG1, IgG2, IgG2, IgG3, IgG4. In a still further specific aspect, the human constant region is IgG1. In a still further aspect, the murine constant region is selected from the group consisting of IgG1, IgG2A, IgG2B, IgG3. In a still further aspect, the murine constant region if IgG2A. In a still further specific aspect, the antibody has reduced or minimal effector function. In a still further specific aspect, the minimal effector function results from production in prokaryotic cells. In a still further specific aspect the minimal effector function results from an “effector-less Fc mutation” or aglycosylation. In still a further embodiment, the effector-less Fc mutation is an N297A or D265A/N297A substitution in the constant region.

In another further embodiment, provided is an isolated anti-PD-L1 antibody comprising a heavy chain and a light chain variable region sequence, wherein:
(a) the heavy chain sequence has at least 85% sequence identity to the heavy chain sequence:

(SEQ ID NO: 7)
EVQLVESGGGLVQPGGSLRLSCAASGFTFSDSWIHW
VRQAPGKGLEWVAWISPYGGSTYYADSVKGRFTISA
DTSKNTAYLQMNSLRAEDTAVYYCARRHWPGGFDYW
GQGTLVTVSS,

or
(b) the light chain sequence has at least 85% sequence identity to the light chain sequence:

(SEQ ID NO: 9)
DIQMTQSPSSLSASVGDRVTITCRASQDVSTAVAWYQQKPG
KAPKLLIYSASFLYSGVPSRFSGSGSGTDFTLTISSLQPED
FATYYCQQYLYHPATFGQGTKVEIKR.

In a still further embodiment, provided is an isolated anti-PD-L1 antibody comprising a heavy chain and a light chain variable region sequence, wherein:
(a) the heavy chain sequence has at least 85% sequence identity to the heavy chain sequence:

(SEQ ID NO: 8)
EVQLVESGGGLVQPGGSLRLSCAASGFTFSDSWIHWVR
QAPGKGLEWVAWISPYGGSTYYADSVKGRFTISADTSK
NTAYLQMNSLRAEDTAVYYCARRHWPGGFDYWGQGTLV
TVSSASTK,

or
(b) the light chain sequences have at least 85% sequence identity to the light chain sequence:

(SEQ ID NO: 9)
DIQMTQSPSSLSASVGDRVTITCRASQDVSTAVAWYQQ
KPGKAPKLLIYSASFLYSGVPSRFSGSGSGTDFTLTIS
SLQPEDFATYYCQQYLYHPATFGQGTKVEIKR.

In a specific aspect, the sequence identity is 86%, 87%, 88%, 89%, 90%, 91%, 92%, 93%, 94%, 95%, 96%, 97%, 98%, 99% or 100%. In another aspect, the heavy chain variable region comprises one or more framework sequences juxtaposed between the HVRs as: (HC-FR1)-(HVR-H1)-(HC-FR2)-(HVR-H2)-(HC-FR3)-(HVR-H3)-(HC-FR4), and the light chain variable regions comprises one or more framework sequences juxtaposed between the HVRs as: (LC-FR1)-(HVR-L1)-(LC-FR2)-(HVR-L2)-(LC-FR3)-(HVR-L3)-(LC-FR4). In yet another aspect, the framework sequences are derived from human consensus framework sequences. In a further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences is the following:

HC-FR1
(SEQ ID NO: 13)
EVQLVESGGGLVQPGGSLRLSCAAS
HC-FR2
(SEQ ID NO: 14)
WVRQAPGKGLEWV
HC-FR3
(SEQ ID NO: 15)
RFTISADTSKNTAYLQMNSLRAEDTAVYYCAR
HC-FR4
(SEQ ID NO: 30)
WGQGTLVTVSS.

In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences is the following:

LC-FR1
(SEQ ID NO: 20)
DIQMTQSPSSLSASVGDRVTITC
LC-FR2
(SEQ ID NO: 21)
WYQQKPGKAPKLLIY
LC-FR3
(SEQ ID NO: 22)
GVPSRFSGSGSGTDFTLTISSLOPEDFATYYC
LC-FR4
(SEQ ID NO: 23)
FGQGTKVEIKR.

In a still further specific aspect, the antibody further comprises a human or murine constant region. In a still further aspect, the human constant region is selected from the group consisting of IgG1, IgG2, IgG2, IgG3, IgG4. In a still further specific aspect, the human constant region is IgG1. In a still further aspect, the murine constant region is selected from the group consisting of IgG1, IgG2A, IgG2B, IgG3. In a still further aspect, the murine constant region if IgG2A. In a still further specific aspect, the antibody has reduced or minimal effector function. In a still further specific aspect, the minimal effector function results from production in prokaryotic cells. In a still further specific aspect the minimal effector function results from an “effector-less Fc mutation” or aglycosylation. In still a further embodiment, the effector-less Fc mutation is an N297A or D265A/N297A substitution in the constant region.

In yet another embodiment, the anti-PD-L1 antibody is atezolizumab, or MPDL3280A (CAS Registry Number: 1380723-44-3). In a still further embodiment, provided is an isolated anti-PD-L1 antibody comprising a heavy chain variable region comprising the heavy chain variable region amino acid sequence from EVQLVESGGGLVQPGGSLRLSCAASGFTFSDSWIHWVRQAPGKGLEWVAWISPYG GSTYYADSVKGRFTISADTSKNTAYLQMNSLRAEDTAVYYCARRHWPGGFDYW GQGTLVTVSS (SEQ ID NO:7) or EVQLVESGGGLVQPGGSLRLSCAASGFTFSDSWIHWVRQAPGKGLEWVAWI SPYGGSTYYADSVKGRFTISADTSKNTAYLQMNSLRAEDTAVYYCARRHWPGGF DYWGQGTLVTVSSASTK (SEQ ID NO:8) and a light chain variable region comprising the amino acid sequence of DIQMTQSPSSLSASVGDRVTITCRASQDVSTAVAWYQQKPGKAPKLLIY SASF LYSGVPSRFSGSGSGTDFTLTISSLQPEDFATYYCQQYLYHPATFGQGTKVEIKR (SEQ ID NO:9). In a still further embodiment, provided is an isolated anti-PD-L1 antibody comprising a heavy chain and/or a light chain sequence, wherein:
(a) the heavy chain sequence has at least 85%, at least 90%, at least 91%, at least 92%, at least 93%, at least 94%, at least 95%, at least 96%, at least 97%, at least 98%, at least 99% or 100% sequence identity to the heavy chain sequence: EVQLVESGGGLVQPGGSLRLSCAASGFTFSDSWIHWVRQAPGKGLEWVA WISPYGGSTYYADSVKGRFTISADTSKNTAYLQMNSLRAEDTAVYYCARR HWPGGFDYWGQGTLVTVSSASTKGPSVFPLAPSSKSTSGGTAALGCLVKD YFPEPVTVSWNSGALTSGVHTFPAVLQSSGLYSLSSVVTVPSSSLGTQTYIC NVNHKPSNTKVDKKVEPKSCDKTHTCPPCPAPELLGGPSVFLFPPKPKDTL MISRTPEVTCVVVDVSHEDPEVKFNWYVDGVEVHNAKTKPREEQYASTYR VVSVLTVLHQDWLNGKEYKCKVSNKALPAPIEKTISKAKGQPREPQVYTLP PSREEMTKNQVSLTCLVKGFYPSDIAVEWESNGQPENNYKTTPPVLDSDGS FFLYSKLTVDKSRWQQGNVFSCSVMHEALHNHYTQKSLSLSPG (SEQ ID NO:31), and/or
(b) the light chain sequences have at least 85%, at least 90%, at least 91%, at least 92%, at least 93%, at least 94%, at least 95%, at least 96%, at least 97%, at least 98%, at least 99% or 100% sequence identity to the light chain sequence:

(SEQ ID NO: 32)
DIQMTQSPSSLSASVGDRVTITCRASQDVSTAVAWYQQKPGKAPKLLIYSA
SFLYSGVPSRFSGSGSGTDFTLTISSLQPEDFATYYCQQYLYHPATFGQGT
KVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGEC.

In a still further embodiment, provided is an isolated nucleic acid encoding a light chain or a heavy chain variable region sequence of an anti-PD-L1 antibody, wherein:
(a) the heavy chain further comprises and HVR-H1, HVR-H2 and an HVR-H3 sequence having at least 85% sequence identity to GFTFSDSWIH (SEQ ID NO:24), AWISPYGGSTYYADSVKG (SEQ ID NO:25) and RHWPGGFDY (SEQ ID NO:12), respectively, and
(b) the light chain further comprises an HVR-L1, HVR-L2 and an HVR-L3 sequence having at least 85% sequence identity to RASQDVSTAVA (SEQ ID NO:26), SASFLYS (SEQ ID NO:27) and QQYLYHPAT (SEQ ID NO:28), respectively.

In a specific aspect, the sequence identity is 86%, 87%, 88%, 89%, 90%, 91%, 92%, 93%, 94%, 95%, 96%, 97%, 98%, 99% or 100%. In one aspect, the heavy chain variable region comprises one or more framework sequences juxtaposed between the HVRs as: (HC-FR1)-(HVR-H1)-(HC-FR2)-(HVR-H2)-(HC-FR3)-(HVR-H3)-(HC-FR4), and the light chain variable regions comprises one or more framework sequences juxtaposed between the HVRs as: (LC-FR1)-(HVR-L1)-(LC-FR2)-(HVR-L2)-(LC-FR3)-(HVR-L3)-(LC-FR4). In yet another aspect, the framework sequences are derived from human consensus framework sequences. In a further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences is the following:

HC-FR1
(SEQ ID NO: 13)
EVQLVESGGGLVQPGGSLRLSCAAS
HC-FR2
SEQ ID NO: 14)
WVRQAPGKGLEWV
HC-FR3
(SEQ ID NO: 15)
RFTISADTSKNTAYLQMNSLRAEDTAVYYCAR
HC-FR4
(SEQ ID NO: 16)
WGQGTLVTVSA.

In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences is the following:

LC-FR1
(SEQ ID NO: 20)
DIQMTQSPSSLSASVGDRVTITC
LC-FR2
(SEQ ID NO: 21)
WYQQKPGKAPKLLIY
LC-FR3
(SEQ ID NO: 22)
GVPSRFSGSGSGTDFTLTISSLQPEDFATYYC
LC-FR4
(SEQ ID NO: 23)
FGQGTKVEIKR.

In a still further specific aspect, the antibody described herein (such as an anti-PD-1 antibody, an anti-PD-L1 antibody, or an anti-PD-L2 antibody) further comprises a human or murine constant region. In a still further aspect, the human constant region is selected from the group consisting of IgG1, IgG2, IgG2, IgG3, IgG4. In a still further specific aspect, the human constant region is IgG1. In a still further aspect, the murine constant region is selected from the group consisting of IgG1, IgG2A, IgG2B, IgG3. In a still further aspect, the murine constant region if IgG2A. In a still further specific aspect, the antibody has reduced or minimal effector function. In a still further specific aspect, the minimal effector function results from production in prokaryotic cells. In a still further specific aspect the minimal effector function results from an “effector-less Fc mutation” or aglycosylation. In still a further aspect, the effector-less Fc mutation is an N297A or D265A/N297A substitution in the constant region.

In a still further aspect, provided herein are nucleic acids encoding any of the antibodies described herein. In some embodiments, the nucleic acid further comprises a vector suitable for expression of the nucleic acid encoding any of the previously described anti-PD-L1, anti-PD-1, or anti-PD-L2 antibodies. In a still further specific aspect, the vector further comprises a host cell suitable for expression of the nucleic acid. In a still further specific aspect, the host cell is a eukaryotic cell or a prokaryotic cell. In a still further specific aspect, the eukaryotic cell is a mammalian cell, such as Chinese Hamster Ovary (CHO).

The antibody or antigen binding fragment thereof, may be made using methods known in the art, for example, by a process comprising culturing a host cell containing nucleic acid encoding any of the previously described anti-PD-L1, anti-PD-1, or anti-PD-L2 antibodies or antigen-binding fragment in a form suitable for expression, under conditions suitable to produce such antibody or fragment, and recovering the antibody or fragment.

In some embodiments, the isolated anti-PD-L1 antibody is aglycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. Removal of glycosylation sites form an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site another amino acid residue (e.g., glycine, alanine or a conservative substitution).

In this regard it is to be noted that the pharmacokinetics of atezolizumab administered as a single agent have been characterized based on clinical data from study PCD4989g and are consistent with a currently ongoing Phase III Study WO29522 in first line treatment of triple negative breast cancer (TNBC). Atezolizumab anti-tumor activity has been observed across doses from 1 to 20 mg/kg. Overall, atezolizumab exhibits pharmacokinetics that are both linear and consistent with typical IgG1 antibodies for doses ≥1 mg/kg every three weeks (q3w). Pharmacokinetic data (Bai S, Jorga K, Xin Y, et al., A guide to rational dosing of monoclonal antibodies, Clin Pharmacokinet 2012; 51:119-35, incorporated by reference herein in its entirety) does not suggest any clinically meaningful differences in exposure following a fixed dose or a dose adjusted for weight. Atezolizumab dosing schedules of q3w and q2w have been tested. A fixed dose of atezolizumab 800 mg every two weeks (q2w) (equivalent to a body weight-based dose of 10 mg/kg q2w) results in equivalent exposure to the Phase III dose of 1200 mg administered every three weeks (q3w). The q3w schedule is being used in multiple Phase III studies of atezolizumab monotherapy across multiple tumor types and the q2w predominantly used in combination with chemotherapy regimens. In Study PCD4989g, the Kaplan-Meier estimated overall 24-week progression-free survival (PFS) rate was 33% (95% CI: 12%, 53%).

The PD-1 axis inhibitor doses of the present disclosure are suitably from about 400 mg to about 1200 mg, from about 600 mg to about 1000 mg, from about 700 mg to about 900 mg, or about 840 mg. In some aspects, the PD-1 axis inhibitor is a PD-L1 inhibitor, and more particularly is atezolizumab, which is administered at a dose of about 840 mg.

In particular embodiments, the PD-1 axis inhibitor, or more particularly the PD-L1 inhibitor, is administered intravenously every 14 days of a 28-day treatment cycle. In some aspects, the subject is treated with the PD-1 axis inhibitor, and more particularly the PD-L1 inhibitor, on days 1 and 15 of the 28-day treatment cycle.

### Cancers Characterized by a Mutated MAPK Signaling Pathway

In some aspects, the cancer having a mutated MAPK signaling pathway for treatment by the methods of the present disclosure is selected from melanoma, lung, breast, colorectal (CRC), bladder, gallbladder, nephroblastoma, gastrointestinal stromal tumor (GIST), prostate, glioblastoma, myeloid leukemia, multiple myeloma, thyroid, biliary, adenocarcinoma, choriocarcinoma, sarcoma, and combinations thereof. In some aspects, the cancer is selected from melanoma, nephroblastoma, GIST, CRC, sarcoma, gallbladder cancer, bladder cancer, and combinations thereof.

In some aspects, the cancer carries a NRAS mutation, a KRAS mutation or a RAF mutation. In some such aspects, the cancer has at least one mutation selected from a BRAF V600E mutation, a KRAS G12V mutation, a KRAS G12D mutation, a KRAS G12C mutation, a KRAS Q61H mutation, a NRAS G13D mutation, a NRAS G12D mutation, a NRAS Q61K mutation, a NRAS Q61R mutation, and a NRAS G12C mutation.

In some aspects, the cancer carries a RAF mutation. In some such aspects, the cancer carries a BRAF V600E mutation. In some such aspects, the cancer is selected from nephroblastoma carrying a BRAF V600E, melanoma carrying a BRAF V600E mutation, GIST carrying a BRAF V600E mutation, CRC carrying a BRAF V600E mutation, and combinations thereof. In other such aspects, the cancer is a melanoma carrying a BRAF V600E mutation, nephroblastoma carrying a BRAF V600E mutation, GIST carrying a BRAF V600E mutation, and combinations thereof. In other such aspect, the cancer is selected from melanoma carrying a BRAF V600E mutation, GIST carrying a BRAF V600E mutation, and combinations thereof. In some such aspects, the melanoma is metastatic or unresectable.

In some NRAS/KRAS aspects, the cancer is metastatic or unresectable melanoma.

In some such aspects, the cancer is melanoma carrying a NRAS mutation.

In some NRAS/KRAS aspects, the cancer is selected from sarcoma carrying a KRAS G12V mutation, melanoma carrying a NRAS G13D mutation, a NRAS G12D mutation, melanoma carrying a NRAS Q61K mutation, melanoma carrying a NRAS Q61R mutation, melanoma carrying a NRAS G12C mutation, gallbladder cancer carrying a KRAS G12D mutation, CRC carrying a KRAS G12C mutation, CRC carrying a KRAS Q61H mutation, CRC carrying a KRAS G12D mutation, bladder cancer carrying a KRAS G12D mutation, bladder cancer carrying a KRAS G12V mutation, and combinations thereof.

In other NRAS/KRAS aspects, the cancer is sarcoma carrying a KRAS G12V mutation, melanoma carrying a NRAS G13D mutation, a NRAS G12D mutation, melanoma carrying a NRAS G12C mutation, gallbladder cancer carrying a KRAS G12D mutation, CRC carrying a KRAS G12C mutation, CRC carrying a KRAS Q61H mutation, CRC carrying a KRAS G12D mutation, bladder cancer carrying a KRAS G12D mutation, bladder cancer carrying a KRAS G12V mutation, and combinations thereof. In some such aspects, the cancer is selected from sarcoma carrying a KRAS G12V mutation, melanoma carrying a NRAS G13D mutation, melanoma carrying a NRAS Q61K mutation, melanoma carrying a NRAS Q61R mutation, and combinations thereof.

### Combination Therapies

The combination therapy of a RAF inhibitor and a PD-1 axis inhibitor targets the MAPK signaling pathway and, based on the present experimental evidence, it is believed that the combination therapy will lead to synergistic anti-tumor activity in cancers characterized by a mutated MAPK signaling pathway. It is still further believed that the combination therapy of the present disclosure may prolong the median progression-free survival time for a subject having such cancer. The present combination therapy is believed to provide particular utility in the treatment of metastatic and/or unresectable melanoma carrying a RAF mutation, such as for instance, a BRAF V600E mutation.

It is further believed that combining a RAF inhibitor, such as belvarafenib, and PD-1 axis inhibitor, such as atezolizumab, will offer to patients with cancer characterized by a mutated MAPK signaling pathways an active treatment with reduced toxicity compared with chemotherapy based regimens. Further, because the mechanism of action of the combination therapy of the present invention differs from the traditional chemotherapy regimens, it is further believed that the activity of further standard therapies will not be significantly affected and will allow patients with progressive disease to continue treatment.

In this regard it is to be noted that any combination of the recited dosages ranges for a recited component of the combination may be used without departing from the intended scope of the present disclosure. In some aspects of the present disclosure, a cancer therapy drug combination is provided comprising: (i) a RAF inhibitor in a dose of from about 250 mg to about 500 mg, or from about 350 mg to about 450 mg of belvarafenib or a pharmaceutically acceptable salt thereof twice per day; and (ii) a PD-1 axis inhibitor in a dose of from about 400 mg to about 1200 mg, from about 600 mg to about 1000 mg, from about 700 mg to about 900 mg, or about 840 mg. In one particular aspect, the RAF inhibitor is belvarafenib and the PD-L1 inhibitor is atezolizumab.

When a subject is administered the drug combination (i.e., the RAF inhibitor and the PD-1 axis inhibitor) on the same day, the drugs may be administered separately in any order. In some aspects, the RAF inhibitor and the PD-1 axis inhibitor are each administered on the same day, and the RAF inhibitor is administered prior to, after, or concurrently with administration of the PD-1 axis inhibitor. Administration of each drug of the drug combination may be separated by some period of time, such as 0.5 hours, 1 hour, 2 hours, 3 hours or 4 hours. In some particular aspects, belvarafenib may be administered orally and atezolizumab may be administered intravenously. In such aspects, belvarafenib may be administered before or after atezolizumab, or they may be administered at the same time or closely spaced in time. In some aspects, the RAF inhibitor and the PD-1 axis inhibitor are each administered on days 1 and 15 of a 28-day treatment cycle, and the RAF inhibitor is administered on days 1 to 21 of the 28-day treatment cycle or on days 1 to 28 of the 28-day treatment cycle.

In some aspects, the RAF inhibitor is administered with food.

In some aspects, the subject was previously administered a course of treatment with an anti-PD-1 drug or anti-PD-L1 drug.

In some aspects, prior to treatment by the method according to the present disclosure, the subject experienced disease progression after treatment with immunotherapy, BRAF V600E therapy, or a combination of immunotherapy and BRAF V600E therapy.

In some aspects, the method for treating cancer is characterized by the absence of the development of squamous cell carcinoma in the human subject.

### EXAMPLES

The Institutional Animal Care and Use Committee of the Hanmi Research Center approved the animal study protocols of the examples.

### Example 1

Example 1 evaluated the efficacy of belvarafenib monotherapy, atezolizumab monotherapy, a combination therapy of belvarafenib and atezolizumab in a mutant K1735 subcutaneous mouse model.

The mouse strain was C3H/HeNCrlOri which is widely used for the K1735 syngeneic model. The mice were supplied by Orient Bio Inc., Korea. The mice were female and were 9-11 weeks of age at the start of dosing, and had a body weight range of 19 to 26 grams.

The cell line was K1735 and was supplied by the American Type Culture Collection (ATCC). The K1735 cells are melanoma cells carrying the NRASG13D mutation. The cell culture in vitro media was Roswell Park Memorial Institute (RPMI) 10% fetal bovine serum (FBS) and incubation was at 5% C02 and 37° C. For the K1735 syngeneic model, cancer cells (1.5×108 cells/10 mL) were mixed with Hank's balanced salt solution (HBSS) and injected subcutaneously at 0.1 mL/head. Seven animals per group were treated with the vehicle (control), belvarafenib monotherapy at 7.5 mg/kg, belvarafenib monotherapy at 15 mg/kg, atezolizumab monotherapy at 10 mg/kg, belvarafenib (7.5 mg/kg) and atezolizumab (10 mg/kg) combination therapy, and belvarafenib (15 mg/kg) and atezolizumab (10 mg/kg) combination therapy. The experiment was done using 1^{st} generation tumor tissue.

The mice were kept in conventional animal lab cages for 14 days for acclimation before the start of the experiment. During the acclimation period, the mice were observed daily with respect to health and any sign of disease. The mice were housed in a clean barrier room in polysulfone cages 1291H (W425×D266×H185 mm, Techniplast, Italy). Ten mice were housed in each cage at a temperature of 22±2° C., a relative humidity of 50±20%, a ventilation frequency of 10-15 times/h, a 12 hour light/dark cycle, a light intensity of 150-300 Lux, at least weekly cage replacement. The mice were fed Picolab Rodent diet (5053, Lab Diet, USA) with abundant tap water.

Belvarafenib dihydrochloride, 99.6% purity, that was stored at room temperature was used. Dosing was based on active ingredient free base and was corrected for assay and water content. The dosing vehicle was DMSO (5%), cremophore EL (5%), and deionized water (90%). Belvarafenib for dosing was dissolved in the vehicle. Belvarafenib oral doses of 7.5 mg/kg and 15 mg/kg were evaluated as monotherapies and in combination with atezolizumab. Belvarafenib was dose daily for 21 days.

Atezolizumab (TECENTRIQ©) that was stored at 2-8° C. was used. The dosing vehicle was saline and the dosing concentration was 5 mL/kg. An atezolizumab intravenous dose of 10 mg/kg was evaluated as a monotherapy and in combination with 7.5 mg/kg and 15 mg/kg belvarafenib. Atezolizumab was dosed three times a week for 3 weeks.

The following observations and measurements were made.

Clinical signs. The general clinical sign and mortality were observed at least once a day during the dosing period.

Body weight. Body weight measurement was performed twice a week during the dosing period.

Relative body weight. Relative body weight (%)=body weight (g)/initial body weight (g)×100.

Tumor size. Tumor size was assessed by digital caliper (MITUTOYO CD-15CPX, MonotaRO Singapore, Japan) twice a week during the dosing period and calculated using the formula for an ellipsoid sphere (V=L×S2/2=mm3, where L=long diameter, and S=short diameter). Tumor size was recorded on the day of the body weight measurement and data was recorded on a tumor volume record sheet.

Relative tumor volume (RTV). RTV(%)=tumor volume/initial tumor volume×100.

Tumor Growth (TG). TG (%)=RTVdayx/RTVday0×100.

Relative tumor growth (RTG). The ratio of mean tumor volume on last measurement day to the mean tumor volume on Day 0.

Inhibition rate (IR). Inhibition of tumor growth was calculated relative to vehicle-treated controls. IR (%)=(1−mean relative tumor weight in treated group/mean relative tumor weight in control group)×100.

Maximum inhibition rate (MIR). MIR was the highest recorded IR (%) among the measured inhibition rate (%).

Mean tumor weight (MTW). MTW was at initiation of study and termination of study.

Maximum weight loss (MWL). MWL was the highest recorded weight loss (%) among the measured weight loss (%).

Flow cytometry was used for tumor infiltrated CD3+CD8+ T cells analysis using BD FACSCanto™ IL (BD Biosciences) and FlowJo™ v 10.6.2 software (BD Biosciences). At the end of the experiment, tumor tissue, excluding mono and combination therapy groups using belvarafenib 7.5 mg/kg, were collected for evaluation of pharmacodynamics. Tumors were surgically dissected, washed twice with phosphate-buffered saline (PBS), and then chopped into 1 mm3 pieces. The chopped tumor pieces were digested in 7 mL of dissociation medium (RPMI medium with 10% FBS, collagenase type II at 2 mg/mL, collagenase type IV at 2 mg/mL, and DNase I at 1 mg/mL) for 30 minutes at 37° C., and filtered through a 70 μm cell strainer (BD Pharm, USA). The filtered cells were then washed twice with PBS. Remaining red blood cells were lysed using ammonium-chloride-potassium (ACK) solution. The cells, which had dissociated into single cells, were stained with Fe block (anti CD16/32, Ref. 14-0161-82, Invitrogen, USA) at 4° C. for 15 minutes to prevent nonspecific antibody binding. The cells were then stained with appropriate antibodies at 4° C. for 30 minutes. To assess the presence of CD3+CD8+ T cell population, cells were stained with PE Cy7-conjugated anti-CD3e (1:100 dilution) (clone: 145-2C11, Invitrogen, USA) and FITC-conjugated anti-CD8a (1:100 dilution) (clone: 53-6.7, Invitrogen, USA).

Statistical analysis of tumor size was performed with GraphPad Prism version 6 (GraphPad software, Inc., USA). Values were expressed as S.E.M. The significance of differences among multiple groups was assessed using a 2-way ANOVA. Post hoc pairwise comparison between groups was tested for significance using Dennett's method.

With the exception of tumor necrosis for one mouse in the seven-mouse group at day 21 of the atezolizumab monotherapy, no clinical signs were observed.

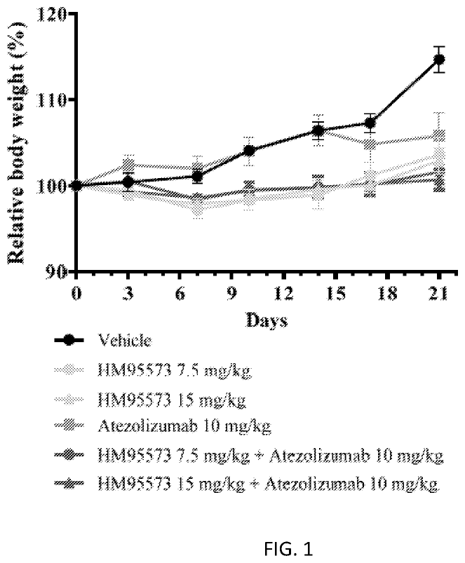

Results are depicted in FIGS. 1 to 3 and in Tables 1 to 3 below.

Body weight loss is depicted in FIG. 1, which is a plot of mouse body weight change versus time for a K1735 syngeneic model for belvarafenib monotherapy (7.5 mg/kg and 15 mg/kg), atezolizumab monotherapy (10 mg/kg), combination therapy at 7.5 mg/kg belvarafenib and 10 mg/kg atezolizumab, and combination therapy at 15 mg/kg belvarafenib and 10 mg/kg atezolizumab of the present disclosure. HM95573 refers to belvarafenib. Point, mean of relative body weight: bars, S.E.M. No specific body weight loss and clinical symptoms were found during the administration.

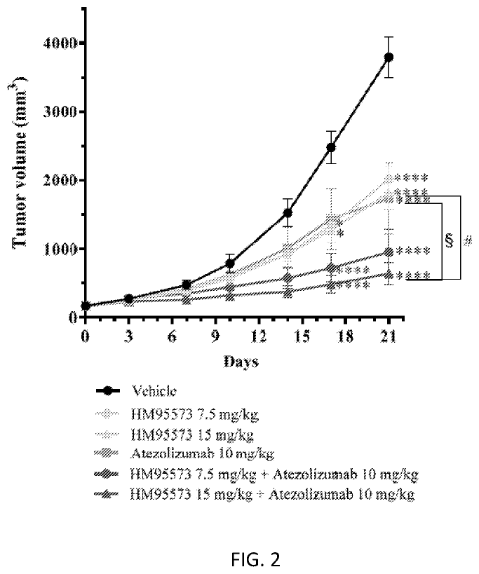

Tumor volume is depicted in FIG. 2, which is a plot of mouse tumor volume versus time for a K1735 syngeneic model for belvarafenib monotherapy (7.5 mg/kg and 15 mg/kg), atezolizumab monotherapy (10 mg/kg), belvarafenib/atezolizumab combination therapy ((i) 7.5 mg/kg and 10 mg/kg and (ii) 15 mg/kg and 10 mg/kg) of the present disclosure. In FIG. 2: HM95573 refers to belvarafenib; * refers to P<0.05; **** refers to P<0.0001; § refers to P<0.05 compared with 10 mg/kg atezolizumab; and # refers to P<0.05 compared with 15 mg/kg belvarafenib. P values were calculated using two-way ANOVA. Point, mean of tumor volume: bars, S.E.M.

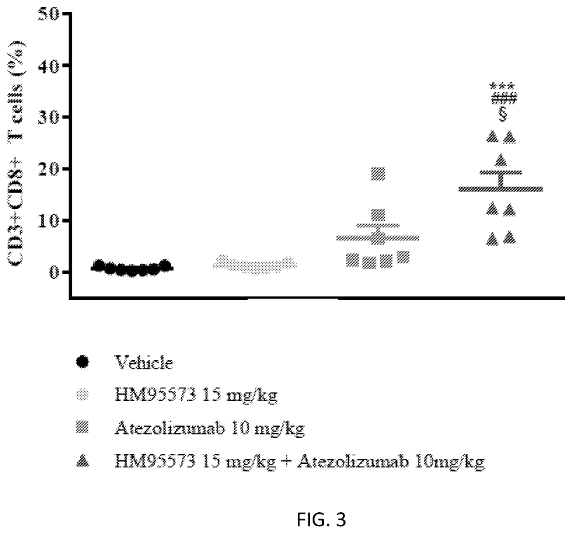

FIG. 3 is a plot of CD3+CD8+ T cells for a K1735 syngeneic mouse model with belvarafenib monotherapy (15 mg/kg), atezolizumab monotherapy (10 mg/kg), and belvarafenib/atezolizumab combination therapy (15 mg/kg and 10 mg/kg). In FIG. 3: HM95573 refers to belvarafenib; *** refers to P<0.001 compared to vehicle control; ### refers to P<0.001 compared with 15 mg/kg belvarafenib; and § refers to P<0.05 compared with atezolizumab 10 mg/kg. P values were calculated using one-way ANOVA. Point, mean of CD3+CD8+ T cells: bars, S.E.M.

Table 1 presents a general summary of the results. The maximum inhibition rate (%) is the highest value among the IR (%). The maximum weight loss (%) is the highest recorded value among the measured body weight loss (%). In Table 1: “Belv. Mono.” refers to belvarafenib monotherapy; “Atezo. Mono.” refers to atezolizumab monotherapy; “Belv./Atezo. Comb.” refers to belvarafenib/atezolizumab combination therapy; “QDx21” refers to a daily oral dose for 21 days; “TIWx3” refers to a three time per week intraperitoneal dose for 21 days; “Max Inhib.” refers to the maximum inhibition rate in %; and “Max Wt.” refers to the maximum weight loss in %.

TABLE 1

| | Dose | | Max | Max |
|---|---|---|---|---|
| Therapy | (mg/kg) | Schedule | Inhib. | Wt. |
| Belv. Mono. | 7.5 | QDx21 | 48.2 | 2.8 |
| Belv. Mono. | 15 | QDx21 | 54.7 | 2.2 |
| Atezo. Mono. | 10 | TIWx3 | 53.4 | — |
| Belv./Atezo. Comb. | 7.5 + 10 | QDx21; TIWx3 | 78.3 | 1.6 |
| Belv./Atezo. Comb. | 15 + 10 | QDx21; TIWx3 | 84.8 | 1.4 |

Table 2 presents the antitumor activity of belvarafenib monotherapy, atezolizumab monotherapy, and belvarafenib+atezolizumab combination therapy in the K1735 syngeneic mouse model. In Table 2: “Belv.” refers to belvarafenib monotherapy; “Atezo.” refers to atezolizumab monotherapy; “Belv.+Atezo.” refers to belvarafenib and atezolizumab combination therapy; “QDx21” refers to a daily oral dose for 21 days; “TIWx3” refers to a three time per week intraperitoneal dose for 21 days; “MTV₀” refers to mean tumor volume on the first day of treatment; “MTV₂₁” refers to mean tumor volume on day 21 of treatment; “RTG” refers to relative tumor growth; “ ” refers to maximum weight loss; “IR (Day 21)” refers to inhibition rate at day 21; and “MIR” refers to maximum inhibition rate.

TABLE 2

| | | | | | Belv. + | Belv. + |
|---|---|---|---|---|---|---|
| | Vehicle | Belv. | Belv. | Atezo. | Atezo. | Atezo. |
| Schedule | QDx21 | QDx21 | QDx21 | TIWx3 | QDx21; | QDx21; |
| | | | | | TIWx3 | TIWx3 |
| Dose | None | 7.5 | 15 | 10 | 7.5 + 10 | 15 + 10 |
| (mg/kg) | | | | | | |
| MTV₀ | 165.9 | 165.8 | 165.9 | 165.0 | 164.6 | 164.5 |
| (mm³) | | | | | | |
| MTV₂₁ | 3791.9 | 2014.8 | 1816.8 | 1733.2 | 950.9 | 635.4 |
| (mm³) | | | | | | |
| RTG | 22.9 | 12.2 | 11.0 | 10.5 | 5.8 | 3.9 |
| MWL (%) | — | 2.8 | 2.2 | — | 1.6 | 1.4 |
| IR (Day 21) | — | 48.2 | 54.7 | 53.4 | 78.3 | 84.8 |
| MIR | — | 48.2 | 54.7 | 53.4 | 78.3 | 84.8 |

Table 3 presents the proportion of CD3+CD8+ T cells of belvarafenib monotherapy, atezolizumab monotherapy, and belvarafenib+atezolizumab combination therapy in the K1735 syngeneic mouse model.

TABLE 3

| | | Dose | CD3⁺CD8⁺ T cells |
|---|---|---|---|
| | Schedule | (mg/kg) | (%) |
| Vehicle | QDx21 | — | 0.65 |
| Belv. | QDx21 | 15 | 1.27 |
| Atezo. | TIWx3 | 10 | 6.52 |
| Belv. + Atezo. | QDx21; TIWx3 | 15 + 10 | 16.0 |

Tumor growth inhibition results. On day 21, belvarafenib (7.5 and 15 mg/kg) and atezolizumab (10 mg/kg) showed 48.2%, 54.7%, and 53.4% tumor growth inhibition, respectively. The combination of belvarafenib (7.5 or 15 mg/kg) and atezolizumab (10 mg/kg) showed 78.3% and 84.8% tumor growth inhibition, respectively, and the combination therapy effects were significant (P<0.05) compared to the monotherapies.

CD3+CD8+ T cell results. Belvarafenib monotherapy (15 mg/kg) and atezolizumab monotherapy (10 mg/kg) showed 1.27% and 6.52% of CD3+CD8+ T cells, respectively, but did not increase significantly as compared to the control group. The combination therapy of belvarafenib (15 mg/kg) and atezolizumab (10 mg/kg) significantly increased CD3+CD8+ T cells to 16.0% compared to each monotherapy (P<0.001 vs. belvarafenib (15 mg/kg) and P<0.05 vs. atezolizumab (10 mg/kg)).

The experimental results show that co-administration of belvarafenib and atezolizumab in a combination therapy synergistically inhibited tumor growth and induced infiltration of cytotoxic T cells as compared to belvarafenib and atezolizumab monotherapies in a NRASG13D mutant K1735 syngeneic mouse model. The results therefore demonstrate that a combination therapy of a pan-RAF inhibitor and a PD-L1 inhibitor may be an effective anticancer therapy in patients with NRASG13D mutant melanoma which have a mutated MAPK signaling system.

### Example 2

Example 2 evaluated the efficacy of belvarafenib monotherapy, Mu igG1 anti-PDL1 monotherapy, and a combination therapy of belvarafenib and Mu igG1 anti-PDL1 in a CT26 syngeneic mouse model (KRASG12D, CRC).

Mu igG1 anti-PDL1 (6E11) WT at 5 mg/kg was dosed orally (PO) twice a week (BIW) for 3 weeks either alone or in combination with belvarafenib. Belvarafenib at 10 mg/kg was dosed PO once a day (QD) for 21 days alone or in combination with Mu igG1 anti-PDL1 (6E11) WT. The vehicle was 5% dimethyl sulfide/5% Cremophor EL (100 μL), 0.5% (w/v) methylcellulose/0.2% Tween 80™.

Tumor volumes were measured in two dimensions (length and width) using Ultra Cal-IV calipers (model 54-10-111; Fred V. Fowler Co.; Newton, MA) and analyzed using Excel, version 14.2.5 (Microsoft Corporation; Redmond WA). The tumor volume was calculated with the following formula: Tumor size (mm3)=(longer measurement×shorter measurement2)×0.5.

$$\\ \% TGI = percent of tumor growth inhibition based on AUC .$$

A generalized additive mixed model (GAMM) was employed to analyze transformed tumor volumes over time as this approach addresses both repeated measurements from the same study subjects and modest dropouts before study end (Lin et al. 1999 and Liang 2005). As tumors generally exhibit exponential growth, tumor volumes were subjected to natural log transformation before analysis. Changes in tumor volumes over time in each group are described by fits (i.e., regression splines with auto-generated spline bases) generated using customized functions in R version 3.4.2 (2017-09-28) (R Development Core Team 2008; R Foundation for Statistical Computing; Vienna, Austria) which integrate software from open source packages including Ime4, mgcv, gamm4, multcomp, settings, plyr, and several packages from the tidyverse such as magrittr, dplyr, tidyr, and ggplot2.

Anti-tumor responses were noted during studies with partial responses (PRs), for purposes of this example, being defined as a >50% decrease from the initial tumor volume and complete responses (CRs), for purposes of this example, being defined as a 100% decrease in tumor volume.

Animal body weights were measured using an Adventura Pro AV812 scale (Ohaus Corporation; Pine Brook, NJ). Percent weight change was calculated using the following formula: Body weight change (%)=[(current body weight/initial body weight)−1)×100]. Percent animal weight was tracked for each individual animal while on study and percent change in body weight for each group was calculated and plotted.

A generalized additive mixed model (GAMM) was also employed to analyze raw body weights (i.e., grams) over time. After data fitting, raw body weight data at each time point from all individual animals and all group fits were normalized and re-plotted separately in two distinct ways: 1) normalized to the starting weight and reported as a percentage to yield % body weight change; and 2) normalized to the maximum weight to date and reported as a percentage to yield % body weight loss.

Estimates of efficacy were obtained by calculating the percent difference between the daily average baseline-corrected AUC of the relevant group fits on the original (i.e., untransformed) scale over a common time period.

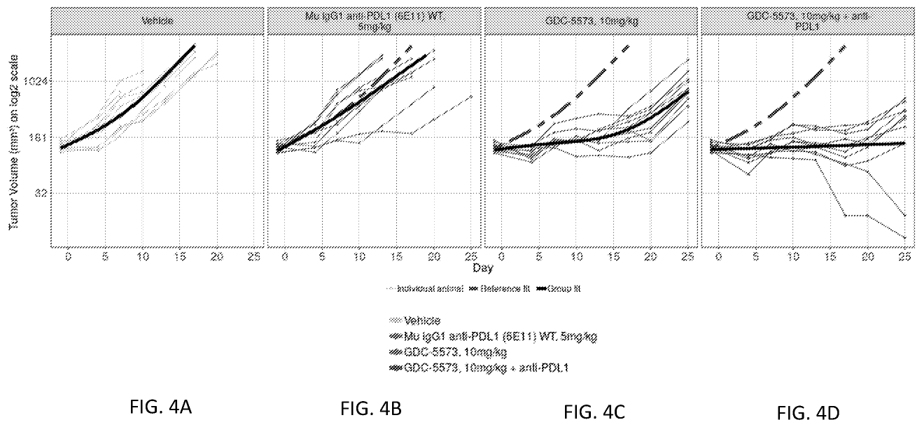

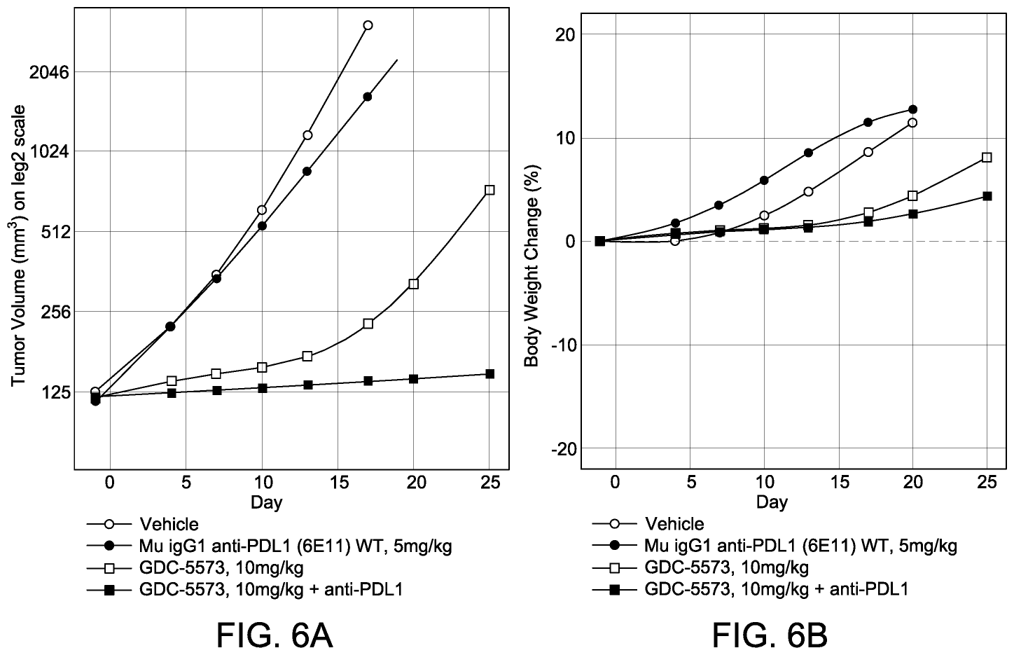

The tumor volume results are depicted in FIG. 4A for the vehicle, FIG. 4B for Mu igG1 anti-PDL1 (6E11) WT, FIG. 4C for Belvarafenib, FIG. 4D for the combination of Mu igG1 anti-PDL1 (6E11) WT and belvarafenib, and FIG. 6A for the associated overlay fits tumor volumes.

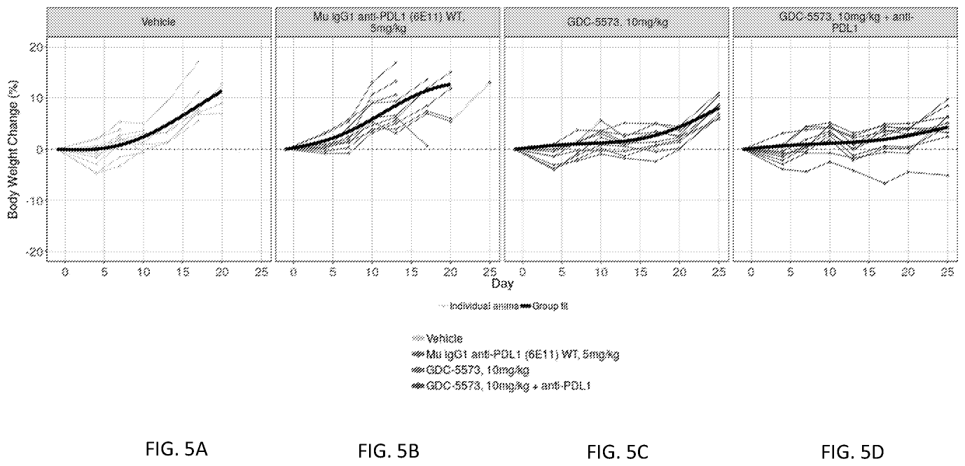

The body weight change results are depicted in FIG. 5A for the vehicle, FIG. 5B for Mu igG1 anti-PDL1 (6E11) WT, FIG. 5C for Belvarafenib, FIG. 5D for the combination of Mu igG1 anti-PDL1 (6E11) WT and belvarafenib, and FIG. 6B for the associated overlay fits body weight change.

### Example 3

The protocol of Example 2 was repeated in an EMT6 syngeneic model (KRASWT, TNBC).

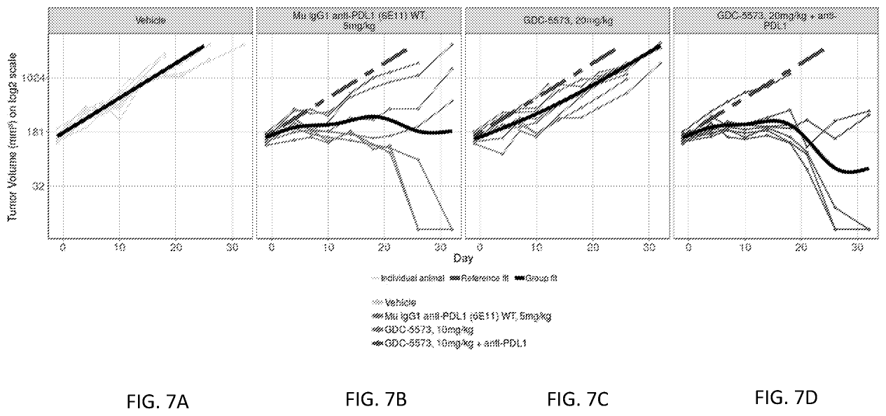

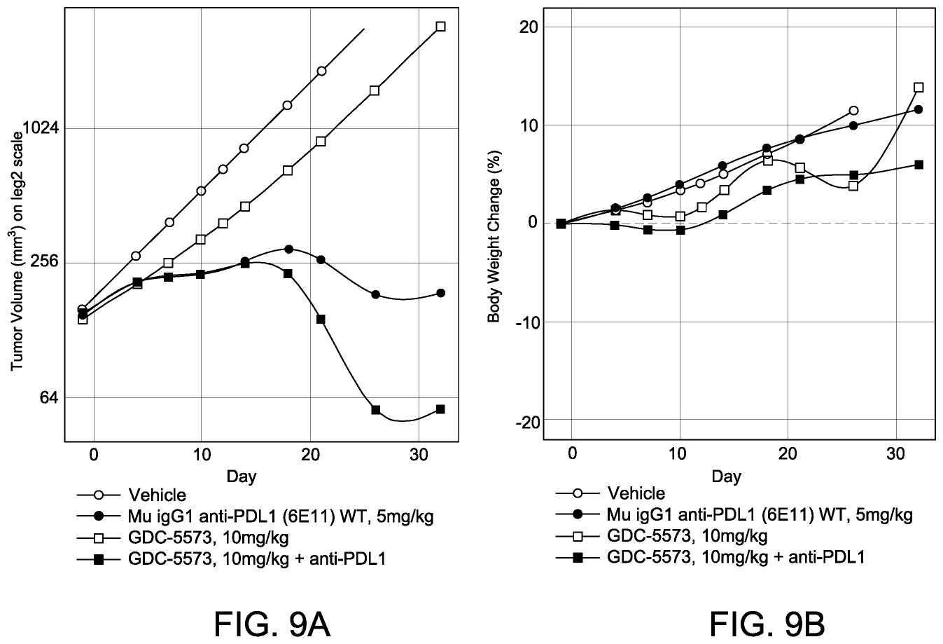

The tumor volume results are depicted in FIG. 7A for the vehicle, FIG. 7B for Mu igG1 anti-PDL1 (6E11) WT, FIG. 7C for Belvarafenib, FIG. 7D for the combination of Mu igG1 anti-PDL1 (6E11) WT and belvarafenib, and FIG. 9A for the associated overlay fits tumor volumes.

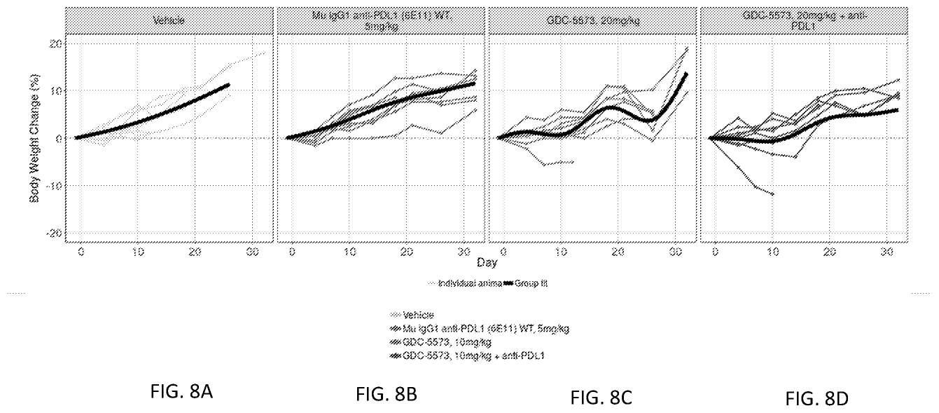

The body weight change results are depicted in FIG. 8A for the vehicle, FIG. 8B for Mu igG1 anti-PDL1 (6E11) WT, FIG. 8C for Belvarafenib, FIG. 8D for the combination of Mu igG1 anti-PDL1 (6E11) WT and belvarafenib, and FIG. 9B for the associated overlay fits body weight change.

This written description uses examples to disclose the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method of treating a subject having cancer characterized by a mutated MAPK signaling pathway, the method comprising: (i) administering to said subject a therapy consisting essentially of (ii) a therapeutically effective amount of a RAF inhibitor and (iii) a therapeutically effective amount of a PD-1 axis inhibitor.

2. The method of claim 1, wherein the cancer is selected from melanoma, lung, breast, colorectal (CRC), bladder, gallbladder, nephroblastoma, gastrointestinal stromal tumor (GIST), prostate, glioblastoma, myeloid leukemia, multiple myeloma, thyroid, biliary, adenocarcinoma, choriocarcinoma, sarcoma, and combinations thereof.

3. The method of claim 2, wherein the cancer is selected from melanoma, nephroblastoma, GIST, CRC, sarcoma, gallbladder cancer, bladder cancer, and combinations thereof.

4. The method of any one of claims 1 to 3, wherein the cancer carries a RAF mutation.

5. The method of claim 4, wherein the cancer carries a BRAF V600E mutation.

6. The method of claim 4 or claim 5, wherein the cancer is selected from nephroblastoma carrying a BRAF V600E, melanoma carrying a BRAF V600E mutation, GIST carrying a BRAF V600E mutation, CRC carrying a BRAF V600E mutation, and combinations thereof.

7. The method of claim 6, wherein the cancer is a melanoma carrying a BRAF V600E mutation, nephroblastoma carrying a BRAF V600E mutation, GIST carrying a BRAF V600E mutation, and combinations thereof.

8. The method of claim 7, wherein the cancer is selected from melanoma carrying a BRAF V600E mutation, GIST carrying a BRAF V600E mutation, and combinations thereof.

9. The method of any one of claims 5 to 8, wherein the melanoma is metastatic or unresectable.

10. The method of any one of claims 1 to 9, wherein the cancer carries a NRAS mutation or a KRAS mutation.

11. The method of claim 10, wherein the cancer has at least one mutation selected from a BRAF V600E mutation, a KRAS G12V mutation, a KRAS G12D mutation, a KRAS G12C mutation, a KRAS Q61H mutation, a NRAS G13D mutation, a NRAS G12D mutation, a NRAS Q61K mutation, a NRAS Q61R mutation, and a NRAS G12C mutation.

12. The method of claim 11, wherein the cancer is selected from sarcoma carrying a KRAS G12V mutation, melanoma carrying a NRAS G13D mutation, melanoma carrying a NRAS G12D mutation, melanoma carrying a NRAS Q61K mutation, melanoma carrying a NRAS Q61R mutation, melanoma carrying a NRAS G12C mutation, gallbladder cancer carrying a KRAS G12D mutation, CRC carrying a KRAS G12C mutation, CRC carrying a KRAS Q61H mutation, CRC carrying a KRAS G12D mutation, bladder cancer carrying a KRAS G12D mutation, bladder cancer carrying a KRAS G12V mutation, and combinations thereof.

13. The method of claim 12, wherein the cancer is sarcoma carrying a KRAS G12V mutation, melanoma carrying a NRAS G13D mutation, melanoma carrying a NRAS G12D mutation, melanoma carrying a NRAS G12C mutation, gallbladder cancer carrying a KRAS G12D mutation, CRC carrying a KRAS G12C mutation, CRC carrying a KRAS Q61H mutation, CRC carrying a KRAS G12D mutation, bladder cancer carrying a KRAS G12D mutation, bladder cancer carrying a KRAS G12V mutation, and combinations thereof.

14. The method of claim 13, wherein the cancer is selected from sarcoma carrying a KRAS G12V mutation, melanoma carrying a NRAS G13D mutation, melanoma carrying a NRAS G12D mutation, melanoma carrying a NRAS Q61K mutation, melanoma carrying a NRAS Q61R mutation, and combinations thereof

15. The method of any one of claims 10 to 14, wherein the cancer is melanoma carrying a NRAS mutation.

16. The method of claim 15, where the melanoma is metastatic or unresectable.

17. The method of any one of claims 1 to 16, wherein the RAF inhibitor is a pan RAF inhibitor.

18. The method of claim 17 wherein the RAF inhibitor is belvarafenib or a pharmaceutically acceptable salt thereof.

19. The method of any one of claims 1 to 18, wherein the PD-1 axis inhibitor is a PD-L1 inhibitor.

20. The method of claim 19, wherein the PD-L1 inhibitor is an antibody comprising a heavy chain comprising HVR-H1 sequence of GFTFSDSWIH (SEQ ID NO:24), HVR-H2 sequence of AWISPYGGSTYYADSVKG (SEQ ID NO:25), and HVR-H3 sequence of RHWPGGFDY (SEQ ID NO:12); and a light chain comprising HVR-L1 sequence of RASQDVSTAVA (SEQ ID NO:26), HVR-L2 sequence of SASFLYS (SEQ ID NO:27), and HVR-L3 sequence of QQYLYHPAT (SEQ ID NO:28).

21. The method of claim 19 wherein the PD-L1 inhibitor is an antibody comprising:
a heavy chain variable region comprising the amino acid sequence of EVQLVESGGGLVQPGGSLRLSCAASGFTFSDSWIHWVRQAPGKGLEWVA WISPYGGSTYYADSVKGRFTISADTSKNTAYLQMNSLRAEDTAVYYCARR HWPGGFDYWGQGTLVTVSS (SEQ ID NO:7) and
a light chain variable region comprising the amino acid sequence of
(SEQ ID NO: 9)
DIQMTQSPSSLSASVGDRVTITCRASQDVSTAVAWYQQKPGKAPKLLIY
SASFLYSGVPSRFSGSGSGTDFTLTISSLQPEDFATYYCQQYLYHPATF
GQGTK VEIKR

22. The method of any one of claims 1 to 19, wherein the PD-L1 inhibitor is atezolizumab.

23. The method of any one of claims 1 to 22, wherein the subject is treated with from about 100 mg to about 1500 mg of the RAF inhibitor per day.

24. The method of claim 23, wherein the RAF inhibitor is belvarafenib or a pharmaceutically acceptable salt thereof, and further wherein the subject is treated with about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, or about 1300 mg of belvarafenib or a pharmaceutically acceptable salt thereof per day.

25. The method of claim 24, wherein the subject is treated with about 250 mg, about 300 mg, about 350 mg, about 400 mg, about 450 mg, or about 500 mg of belvarafenib or a pharmaceutically acceptable salt thereof twice per day.

26. The method of claim 24 or claim 25, wherein belvarafenib is administered daily for 28 consecutive days of a 28-day treatment cycle.

27. The method of any one of claims 1 to 26, wherein the subject is treated with from about 400 mg to about 1200 mg of the PD-1 axis inhibitor for two days of a 28-day treatment cycle.

28. The method of claim 27, wherein the PD-1 axis inhibitor is Atezolizumab, and further wherein the subject is treated with about 840 mg for two days of a 28-day treatment cycle.

29. The method of any one of claims 1 to 28, wherein the subject is treated with the PD-1 axis inhibitor every 14 days of a 28-day treatment cycle.

30. The method of claim 29, wherein the subject is treated with the PD-1 axis inhibitor on days 1 and 15 of the 28-day treatment cycle.

31. The method of any one of claims 1 to 30, wherein the RAF inhibitor and the PD-1 axis inhibitor are each administered on day 1 and on day 15 of a 28-day treatment cycle.

32. The method of any one of claims 1 to 31, wherein, when the RAF inhibitor and the PD-1 axis inhibitor are each administered on the same day, and the RAF inhibitor is administered prior to, after, or concurrently with administration of the PD-1 axis inhibitor.

33. The method of any one of claims 1 to 32, wherein the RAF inhibitor is administered with food.

34. The method of any one of claims 1 to 33, wherein the subject was previously administered a course of treatment with an anti-PD-1 drug or anti-PD-L1 drug.

35. The method of any one of claims 1 to 34, wherein said method for treating cancer is characterized by the absence of the development of squamous cell carcinoma in the human subject.

36. The method of any one of claims 1 to 35,
wherein the cancer is melanoma, and
wherein, prior to said treatment, the human subject experienced disease progression after treatment with immunotherapy, BRAF V600E therapy, or a combination of immunotherapy and BRAF V600E therapy.
